SEQUENCE LISTING (13 sequences)

SEQ ID NO: 1 - protein, 313 aa, Homo sapiens
MLLSKINSLAHLRAAPCNDLHATKLAPGKEKEPLESQYQVGPLLGSGGFGSVYSGIRVSDNLPVAIKHVEKDRISDWGELPNGTRVPMEVVLLKKVSSGFSGVIRLLDWFERPDSFVLILERPEPVQDLFDFITERGALQEELARSFFWQVLEAVRHCHNCGVLHRDIKDENILIDLNRGELKLIDFGSGALLKDTVYTDFDGTRVYSPPEWIRYHRYHGRSAAVWSLGILLYDMVCGDIPFEHDEEIIRGQVFFRQRVSSECQHLIRWCLALRPSDRPTFEEIQNHPWMQDVLLPQETAEIHLHSLSPGPSK

SEQ ID NO: 2 - protein, 404 aa, Homo sapiens
  misc_feature (214)..(214): Xaa can be any naturally occurring amino acid
MPHEPHEPLTPPFSALPDPAGAPSRRQSRQRPQLSSDSPSAFRASRSHSRNATRSHSHSHSPRHSLRHSPGSGSCGSSSGHRPCADILEVGMLLSKINSLAHLRAAPCNDLHATKLAPGKEKEPLESQYQVGPLLGSGGFGSVYSGIRVSDNLPVAIKHVEKDRISDWGELPNGTRVPMEVVLLKKVSSGFSGVIRLLDWFERPDSFVLILERXEPVQDLFDFITERGALQEELARSFFWQVLEAVRHCHNCGVLHRDIKDENILIDLNRGELKLIDFGSGALLKDTVYTDFDGTRVYSPPEWIRYHRYHGRSAAVWSLGILLYDMVCGDIPFEHDEEIIRGQVFFRQRVSSECQHLIRWCLALRPSDRPTFEEIQNHPWMQDVLLPQETAEIHLHSLSPGPSK

SEQ ID NO: 3 - DNA, 5826 bp, Homo sapiens
cgcgcgcggcgaatctcaacgctgcgccgtctgcgggcgcttccgggccaccagtttctctgctttccaccctggcgccccccagccctggctccccagctgcgctgccccgggcgtccacgccctgcgggcttagcgggttcagtgggctcaatctgcgcagcgccacctccatgttgaccaagcctctacaggggcctcccgcgccccccgggacccccacgccgccgccaggtgagtacatcctcccctactgcaaccagacggggtgggctggaatgatgggttgcagcgcggggggagggagtcgtggctgggctcagcacgccgccaccctgacttcctcgcctccgcctgcgtaggaggcaaggatcgggaagcgttcgaggccgagtatcgactcggccccctcctgggtaaggggggctttggcaccgtcttcgcaggacaccgcctcacagatcgactccaggtatccgtcatgagggtcttgggagggtcaggtgcgtgtggcgggggcgggggtcctggccctggaatgctggttgaccgaggagtgagcctgcagagtgtgtagaggaccaggtgtgtgtgtgtgtgtgtccgtgtccgtgtccgaggagtgagcctgcagtgtgtgtagagggccaggtgtgtgtgcgtgcgcgtgtgtgtgtcggtctaggaggttatgggcggggggggggggcagggggcttcagattccggagttccttgaccccggggtccaggctgtgtatgtgtgggaaagcagggacctagatgtgagatttgtgggacttttggaggtaggtgtccagtgtggagtcatgcggaccaggaccctggtacagagttggggtgtcgtagagctaaataggaagattgtgggcctggggtatcaggaaatctagaactcaggacttggagtgatgagtcctgatgcctgagaacggagagcccagggctaaggaaggtgggagagataaacttggttccgaggacctggagggcaggggagacgccctggtacgcgttctgtggggtgctgtggttggggaccagaaagactagagtgctggtagatggaggaatactggaggtaggcagaaggtctagactgggaggggtctggggatcacctgctggcctccttatcacggccttcttctccaggtggccatcaaagtgattccccggaatcgtgtgctgggctggtcccccttggtgagtaccttcggagcccttcctaacctacctactccatcactgatgtattcacctccttgcttttccaggggatgtatgactccctgggccctgtaacagtgagaatactgccagtccatttatactcccttggggtgacatacagttctgattcaccccaattcccctagagccctggattctcccctccaacaaacctttaccatccttcctccaaacactgctgggggactgcccgcagggcgtgctggtggggaacaaggggcagaggtcactggttgccatggtgatggtggctgcttctctcttgccgttataacgctaacggacatcagggcgggtctgggcaagttgtagagttgggagcgccccctggcgggctctaggggaaactgcgcctgcgcagtccatgggacccaaagggagagggtgcgcctgcgcaatatcggtatttttgcatctcggtgagaaaacgtctgctgccgtgcaagtcagcagcctggccaggagagggctctacctcatcccagaaggttgctgctcgaagtgtacctgcgcagggcttggggaggcagtggggggcggattttgtggcccccagcgtttatacttttttttttttggagacacagtctccctctgttgcccaggctggagtgaggtgacgcgatctcggctcactgcaacctccgtctcctgggttcaagtgattctcctgcctcagcctcccaagtagctgggactacaggagcgcacaaccatgcccggctaatttttgtatttttagtagagacagggtttcaccatgttggccaggcgggttttgaactgctgacctcaggtgatccgcctgcctcggccactcaaagtgctgggattacaggcatgagccaccacgcccggctgcatttatgacttttttttttccttgagacggagtttcgctctgctgcctgggctggagtgcagtggcgtgatctcagctcactgcagcctccacctcctgggttcaagcgattctcctgcctcaggctcctgagtagctggaattacaggcacccgctgccatgcccggctaagttttacgtttttagtagagaccgtgtttcaccatgttggccaggctggtctcgaacccctgacctagtgatctgcccgccttgggcctcccaaagtgctgggattacaggcgtgagccaccgcgcccagcctctaattttgtatttttagtagagacggggtttctccatgttggtcaggctggtctcgaactcccgacctcaggtgatctgcccgtctcggcctcccaaagtgctgggattacaggcgtgagccactgcgcagggccacatttaggctttttattggctggttctaggtgcttggtgatgctgacaaaacacatgataacactaagtccttttgtgctaggtcctttgtaataaatcactcagctgtttaacaaattaggtatattgaccacctactatatgacagacataattctagacactcagcaaagtattacataagtattgagagctcattttgtgctaggtccttttttactaattgttttcacctgtttaacaaatatttattcagccctactctgttagcagccactgttctagtgcttcatatacgtccgtgaacaaaacaaaccattacacaataagtgtttattgagtgctaactgcttgtcagagcccatgctattaagtgctgtcatctgtttaacatttattgatcacctgtgtaaggtactattctaatctgggatatgtcagggaacaaaacaaaacacataatggtggtgctgcttctgctgaaagccttcagttgataaccagatttttctttgtatttttgcttgtttgttttgagacagctggagtgcagtggtgtgatcttcactgcaacctctgccttcttggctcaagcgaccctcccacctgagcctcccaagtagctgggactacaggtgcatgccaccaagcctggctaatttttgtgtttgtgccattttgcccaggctgatcttgaactcttgggctcaagcaatccacccacatcagcctcccaaagtgctgggattgcagggatgagccactgtgcctggccgaacttctttcgtttattcaaatgtttattgatctacgacatgcgagatttgtgcaggctctttgctggtttcaccctctcaatcgctgtgtgagtttgtgtctttagggaaagtgaggcccaggaagggaagtgagttgcttagcgacacactgtcaggaaaaggggccctgagttgagcttaggtaaaaagcctcagagctgttgccctgacatctgtcttttttctctccctgcttcccaccccacctgtgcccccagtcagactcagtcacatgcccactcgaagtcgcactgctatggaaagtgggtgcaggtggtgggcaccctggcgtgatccgcctgcttgactggtttgagacacaggagggcttcatgctggtcctcgagcggcctttgcccgcccaggatctctttgactatatcacagagaagggcccactgggtgaaggcccaagccgctgcttctttggccaagtagtggcagccatccagcactgccattcccgtggagttgtccatcgtgacatcaaggatgagaacatcctgatagacctacgccgtggctgtgccaaactcattgattttggttctggtgccctgcttcatgatgaaccctacactgactttgatggtaaggcttctctaaatctccctggagggattgtttttacttgatggccttgtgacctttggcctccagtggtggggtgtcctgtaatccttgacccatactgcattatataagatgatcgattgctaatactggggattctcagccttgccctctgataaagtccatcttttaatggtgtgctaaccttattctgggctcctattctggtgaggggatcctgttaccatcctgagtattctttctctggtaaggggatcctgttacttttcagtgcttttattctgttgaggggactctgttattttagctgctttttatctagtgaggggactctgcttttatcttgagtgctcttaattgtggtgaggccatccttcctggagagtttggggttggagaagggcatcatgagattgagttggtctaacccctggcttgtgtgcagggacaagggtgtacagccccccagagtggatctctcgacaccagtaccatgcactcccggccactgtctggtcactgggcatcctcctctatgacatggtgtgtggggacattccctttgagagggaccaggagattctggaagctgagctccacttcccagcccatgtctccccaggtgaggcctcactgaccccagcccagaagactccatccttctcagggaccagtaccccctactgactgctaatcttccctctctgcttcttggcctacagactgctgtgccctaatccgccggtgcctggcccccaaaccttcttcccgaccctcactggaagagatcctgctggacccctggatgcaaacaccagccgaggatgtacccctcaacccctccaaaggaggccctgcccctttggcctggtccttgctaccctaagcctggcctggcctggcctggcccccaatggtcagaagagccatcccatggccatgtcacagggatagatggacatttgttgacttggttttacaggtcattaccagtcattaaagtccagtattactaaggtaagggattgaggatcaggggttagaagacataaaccaagtctgcccagttcccttcccaatcctacaaaggagccttcctcccagaacctgtggtccctgattctggagggggaacttcttgcttctcattttgctaaggaagtttattttggtgaagttgttcccattctgagccccgggactcttattctgatgatgtgtcaccccacattggcacctcctactaccaccacacaaacttagttcatatgctcttacttgggcaagggtgctttccttccaataccccagtagcttttattttagtaaagggaccctttcccctagcctagggtcccatattgggtcaagctgcttacctgcctcagcccaggattctttattctgggggaggtaatgccctgttgttaccccaaggcttcttttttttttttttttttttgggtgaggggaccctactctgttatcccaagtgctcttattctggtgagaagaaccttacttccataatttgggaaggaatggaagatggacaccaccggacaccaccagacactaggatgggatggatggttttttgggggatgggctaggggaaataaggcttgctgtttgttctcctggggcgctccctccaacttttgcagattcttgcaacctcctcctgagccgggattgtccaattactaaaatgtaaataatcacgtattgtggggaggggagttccaagtgtgccctcctctcttctcctgcctggattatttaaaaagccatgtgtggaaacccactatttaataaaagtaatagaatcagaa

SEQ ID NO: 4 - DNA, 2795 bp, Homo sapiens
  CDS (431)..(1372)
ccctttactcctggctgcggggcgagccgggcgtctgctgcagcggccgcggtggctgaggaggcccgagaggagtcggtggcagcggcggcggcgggaccggcagcagcagcagcagcagcagcagcagcaaccactagcctcctgccccgcggcgctgccgcacgagccccacgagccgctcaccccgccgttctcagcgctgcccgaccccgctggcgcgccctcccgccgccagtcccggcagcgccctcagttgtcctccgactcgccctcggccttccgcgccagccgcagccacagccgcaacgccacccgcagccacagccacagccacagccccaggcatagccttcggcacagccccggctccggctcctgcggcagctcctctgggcaccgtccctgcgccgacatcctggaggttgggatgctcttgtccaaaatcaactcgcttgcccacctgcgcgccgcgccctgcaacgacctgcacgccaccaagctggcgcccggcaaggagaaggagcccctggagtcgcagtaccaggtgggcccgctactgggcagcggcggcttcggctcggtctactcaggcatccgcgtctccgacaacttgccggtggccatcaaacacgtggagaaggaccggatttccgactggggagagctgcctaatggcactcgagtgcccatggaagtggtcctgctgaagaaggtgagctcgggtttctccggcgtcattaggctcctggactggttcgagaggcccgacagtttcgtcctgatcctggagaggcccgagccggtgcaagatctcttcgacttcatcacggaaaggggagccctgcaagaggagctggcccgcagcttcttctggcaggtgctggaggccgtgcggcactgccacaactgcggggtgctccaccgcgacatcaaggacgaaaacatccttatcgacctcaatcgcggcgagctcaagctcatcgacttcgggtcgggggcgctgctcaaggacaccgtctacacggacttcgatgggacccgagtgtatagccctccagagtggatccgctaccatcgctaccatggcaggtcggcggcagtctggtccctggggatcctgctgtatgatatggtgtgtggagatattcctttcgagcatgacgaagagatcatcaggggccaggttttcttcaggcagagggtctcttcagaatgtcagcatctcattagatggtgcttggccctgagaccatcagataggccaaccttcgaagaaatccagaaccatccatggatgcaagatgttctcctgccccaggaaactgctgagatccacctccacagcctgtcgccggggcccagcaaatagcagcctttctggcaggtcctcccctctcttgtcagatgcccgagggaggggaagcttctgtctccagcttcccgagtaccagtgacacgtctcgccaagcaggacagtgcttgatacaggaacaacatttacaactcattccagatcccaggcccctggaggctgcctcccaacagtggggaagagtgactctccaggggtcctaggcctcaactcctcccatagatactctcttcttctcataggtgtccagcattgctggactctgaaatatcccgggggtggggggtgggggtgggtcagaaccctgccatggaactgtttccttcatcatgagttctgctgaatgccgcgatgggtcaggtaggggggaaacaggttgggatgggataggactagcaccattttaagtccctgtcacctcttccgactctttctgagtgccttctgtggggactccggctgtgctgggagaaatacttgaacttgcctcttttacctgctgcttctccaaaaatctgcctgggttttgttccctatttttctctcctgtcctccctcaccccctccttcatatgaaaggtgccatggaagaggctacagggccaaacgctgagccacctgcccttttttctgcctcctttagtaaaactccgagtgaactggtcttcctttttggtttttacttaactgtttcaaagccaagacctcacacacacaaaaaatgcacaaacaatgcaatcaacagaaaagctgtaaatgtgtgtacagttggcatggtagtatacaaaaagattgtagtggatctaatttttaagaaattttgcctttaagttattttacctgtttttgtttcttgttttgaaagatgcgcattctaacctggaggtcaatgttatgtatttatttatttatttatttggttcccttcctattccaagcttccatagctgctgccctagttttctttcctcctttcctcctctgacttggggaccttttgggggagggctgcgacgcttgctctgtttgtggggtgacgggactcaggcgggacagtgctgcagctccctggcttctgtggggcccctcacctacttacccaggtgggtcccggctctgtgggtgatggggaggggcattgctgactgtgtatataggataattatgaaaagcagttctggatggtgtgccttccagatcctctctggggctgtgttttgagcagcaggtagcctgctggttttatctgagtgaaatactgtacaggggaataaaagagatcttatttttttttttatacttggcgttttttgaataaaaaccttttgtcttaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - protein, 313 aa, Homo sapiens; identical to SEQ ID NO: 1

SEQ ID NO: 6 - protein, 44 aa, Homo sapiens
MLLSKFGSLAHLCGPGGVDHLPVKILQPAKADKESFEKAYQVGA

SEQ ID NO: 7 - protein, 282 aa, Homo sapiens
VLGSGGFGTVYAGSRIADGLPVAVKHVVKERVTEWGSLGGATVPLEVVLLRKVGAAGGARGVIRLLDWFERPDGFLLVLERPEPAQDLFDFITERGALDEPLARRFFAQVLAAVRHCHSCGVVHRDIKDENLLVDLRSGELKLIDFGSGALLKDTVYTDFDGTRVYSPPEWIRYHRYHGRSATVWSLGVLLYDMVCGDIPFEQDEEILRGRLLFRRRVSPECQQLIRWCLSLRPSERPSLDQIAAHPWMLGADGGAPESCDLRLCTLDPDDVASTTSSSESL

SEQ ID NO: 8 - DNA, 2410 bp, Homo sapiens
  CDS (454)..(1434)
gagagcgtgagcgcggagagcggaccgacgcgacacgccgtgcgcctccgcggctgcgctacgaaaacgagtcccggagcggccccgcgcccgccgcacccggccctcgcccgcccgaagacaggcgccaagctgccccgccgtctccccagctagcgcccggccgccgccgcctcgcgggccccgggcggaagggggcggggtcccgattcgccccgcccccgcggagggatacgcggcgccgcggcccaaaacccccgggcgaggcggccggggcgggtgaggcgctccgcctgctgcgcgtctacgcggtccccgcgggccttccgggcccactgcgccgcgcggaccgcctcgggctcggacggccggtgtccccggcgcgccgctcgcccggatcggccgcggcttcggcgcctggggctcggggctccggggaggccgtcgcccgcgatgctgctctccaagttcggctccctggcgcacctctgcgggcccggcggcgtggaccacctcccggtgaagatcctgcagccagccaaggcggacaaggagagcttcgagaaggcgtaccaggtgggcgccgtgctgggtagcggcggcttcggcacggtctacgcgggtagccgcatcgccgacgggctcccggtggctgtgaagcacgtggtgaaggagcgggtgaccgagtggggcagcctgggcggcgcgaccgtgcccctggaggtggtgctgctgcgcaaggtgggcgcggcgggcggcgcgcgcggcgtcatccgcctgctggactggttcgagcggcccgacggcttcctgctggtgctggagcggcccgagccggcgcaggacctcttcgactttatcacggagcgcggcgccctggacgagccgctggcgcgccgcttcttcgcgcaggtgctggccgccgtgcgccactgccacagctgcggggtcgtgcaccgcgacattaaggacgaaaatctgcttgtggacctgcgctccggagagctcaagctcatcgacttcggttcgggtgcgctgctcaaggacacggtctacaccgacttcgacggcacccgagtgtacagccccccggagtggatccgctaccaccgctaccacgggcgctcggccaccgtgtggtcgctgggcgtgcttctctacgatatggtgtgtggggacatccccttcgagcaggacgaggagatcctccgaggccgcctgctcttccggaggagggtctctccagagtgccagcagctgatccggtggtgcctgtccctgcggccctcagagcggccgtcgctggatcagattgcggcccatccctggatgctgggggctgacgggggcgtcccggagagctgtgacctgcggctgtgcaccctcgaccctgatgacgtggccagcaccacgtccagcagcgagagcttgtgaggagctgcacctgactgggagctaggggaccacctgccttggccagacctgggacgcccccagaccctgactttctcctgcgtgggccgtctcctcctgcggaagcagtgacctctgacccctggtgaccttcgctttgagtgccttttgaacgctggtcccgcgggacttggttttctcaagctctgtctgtccaaagacgctccggtcgaggtcccgcctgccctgggtggatacttgaaccccagacgcccctctgtgctgctgtgtccggaggcggccttcccatctgcctgcccacccggagctctttccgccggcgcagggtcccaagcccacctcccgccctcagtcctgcggtgtgcgtctgggcacgtcctgcacacacaatgcaagtcctggcctccgcgcccgcccgcccacgcgagccgtacccgccgccaactctgttatttatggtgtgaccccctggaggtgccctcggcccaccggggctatttattgtttaatttatttgttgaggttatttcctctgagcagtctgcctctcccaagccccaggggacagtggggaggcaggggagggggtggctgtggtccagggaccccaggccctgattcctgtgcctggcgtctgtcccggccccgcctgtcagaagatgaacatgtatagtggctaacttaaggggagtgggtgaccctgacacttccaggcactgtgcccagggtttgggttttaaattattgactttgtacagtctgcttgtgggctctgaaagctggggtggggccagagcctgagcgtttaatttattcagtacctgtgtttgtgtgaatgcggtgtgtgcaggcatcgcagatgggggttctttcagttcaaaagtgagatgtctggagatcatatttttttatacaggtatttcaattaaaatgtttttgtacataaaaaaaaaaaaaaaaaaaaaaaaaaaaaaaa

SEQ ID NO: 9 - protein, 326 aa, Homo sapiens
MLLSKFGSLAHLCGPGGVDHLPVKILQPAKADKESFEKAYQVGAVLGSGGFGTVYAGSRIADGLPVAVKHVVKERVTEWGSLGGATVPLEVVLLRKVGAAGGARGVIRLLDWFERPDGFLLVLERPEPAQDLFDFITERGALDEPLARRFFAQVLAAVRHCHSCGVVHRDIKDENLLVDLRSGELKLIDFGSGALLKDTVYTDFDGTRVYSPPEWIRYHRYHGRSATVWSLGVLLYDMVCGDIPFEQDEEILRGRLLFRRRVSPECQQLIRWCLSLRPSERPSLDQIAAHPWMLGADGGVPESCDLRLCTLDPDDVASTTSSSESL

SEQ ID NO: 10 - DNA, 992 bp, Homo sapiens
agcttcgaattatgctcttgtccaaaatcaactcgcttgcccacctgcgcgccgcgccctgcaacgacctgcacgccaccaagctggcgcccggcaaggagaaggagcccctggagtcgcagtaccaggtgggcccgctactgggcagcggcggcttcggctcggtctactcaggcatccgcgtctccgacaacttgccggtggccatcaaacacgtggagaaggaccggatttccgactggggagagctgcctaatggcactcgagtgcccatggaagtggtcctgctgaagaaggtgagctcgggtttctccggcgtcattaggctcctggactggttcgagaggcccgacagtttcgtcctgatcctggagaggcccgagccggtgcaagatctcttcgacttcatcacggaaaggggagccctgcaagaggagctggcccgcagcttcttctggcaggtgctggaggccgtgcggcactgccacaactgcggggtgctccaccgcgacatcaaggacgaaaacatccttatcgacctcaatcgcggcgagctcaagctcatcgacttcgggtcgggggcgctgctcaaggacaccgtctacacggacttcgatgggacccgagtgtatagccctccagagtggatccgctaccatcgctaccatggcaggtcggcggcagtctggtccctggggatcctgctgtatgatatggtgtgtggagatattcctttcgagcatgacgaagagatcatcaggggccaggttttcttcaggcagagggtctcttcagaatgtcagcatctcattagatggtgcttggccctgagaccatcagataggccaaccttcgaagaaatccagaaccatccatggatgcaagatgttctcctgccccaggaaactgctgagatccacctccacagcctgtcgccggggcccagcagcctgtcgccggggcccagcaaacaattggtaccgcgggcccgg

SEQ ID NO: 11 - DNA, 942 bp, Homo sapiens
atgctcttgtccaaaatcaactcgcttgcccacctgcgcgccgcgccctgcaacgacctgcacgccaccaagctggcgcccggcaaggagaaggagcccctggagtcgcagtaccaggtgggcccgctactgggcagcggcggcttcggctcggtctactcaggcatccgcgtctccgacaacttgccggtggccatcaaacacgtggagaaggaccggatttccgactggggagagctgcctaatggcactcgagtgcccatggaagtggtcctgctgaagaaggtgagctcgggtttctccggcgtcattaggctcctggactggttcgagaggcccgacagtttcgtcctgatcctggagaggcccgagccggtgcaagatctcttcgacttcatcacggaaaggggagccctgcaagaggagctggcccgcagcttcttctggcaggtgctggaggccgtgcggcactgccacaactgcggggtgctccaccgcgacatcaaggacgaaaacatccttatcgacctcaatcgcggcgagctcaagctcatcgacttcgggtcgggggcgctgctcaaggacaccgtctacacggacttcgatgggacccgagtgtatagccctccagagtggatccgctaccatcgctaccatggcaggtcggcggcagtctggtccctggggatcctgctgtatgatatggtgtgtggagatattcctttcgagcatgacgaagagatcatcaggggccaggttttcttcaggcagagggtctcttcagaatgtcagcatctcattagatggtgcttggccctgagaccatcagataggccaaccttcgaagaaatccagaaccatccatggatgcaagatgttctcctgccccaggaaactgctgagatccacctccacagcctgtcgccggggcccagcaaatag

SEQ ID NO: 12 - DNA, 942 bp, Mus musculus
atgctcctgtccaagatcaactccctggcccacctgcgcgccgcgccctgcaacgacctgcacgccaccaagctggcgccgggcaaagagaaggagcccctggagtcgcagtaccaggtgggcccgctgttgggcagcggtggcttcggctcggtctactctggcatccgcgtcgccgacaacttgccggtggccattaagcacgtggagaaggaccggatttccgattggggagaactgcccaatggcacccgagtgcccatggaagtggtcctgttgaagaaggtgagctcggacttctcgggcgtcattagacttctggactggttcgagaggcccgatagtttcgtgctgatcctggagaggcccgaaccggtgcaagacctcttcgactttatcaccgaacgaggagccctacaggaggacctggcccgaggattcttctggcaggtgctggaggccgtgcggcattgccacaactgcggggttctccaccgcgacatcaaggacgagaacatcttaatcgacctgagccgcggcgaaatcaaactcatcgacttcgggtcgggggcgctgctcaaggacacagtctacacggactttgatgggacccgagtgtacagtcctccagagtggattcgctaccatcgctaccacggcaggtcggcagctgtctggtcccttgggatcctgctctatgacatggtctgcggagatattccgtttgagcacgatgaagagatcatcaagggccaagtgttcttcaggcaaactgtctcttcagagtgtcagcaccttattaaatggtgcctgtccctgagaccatcagatcggccctcctttgaagaaatccggaaccatccatggatgcagggtgacctcctgccccaggcagcttctgagatccatctgcacagtctgtcaccggggtccagcaagtag

SEQ ID NO: 13 - DNA, 10570 bp, artificial sequence
  lentiviral construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccgatccacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggcctgcagagatccagagttaggcagggacattcaccattatcgtttcagacccacctcccaaccccggtcatatgggaatgaaagaccccacctgtaggtttggcaagctaggatcaaggttaggaacagagagacagcagaatatgggccaaacaggatatctgtggtaagcagttcctgccccggctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagatcagttaattaagaattcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtttctagagttaactcgagggatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgagacctagaaaaacatggagcaatcacaagtagcaacacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2015/0079049 A1
(43) Pub. Date: Mar. 19, 2015

(54) COMPOSITIONS AND METHODS FOR KINASE-MEDIATED CYTOPROTECTION AND ENHANCED CELLULAR ENGRAFTMENT AND PERSISTENCE

(71) Applicant: San Diego State University (SDSU) Foundation, dba San Diego State University (SDSU) Research Founda, San Diego, CA (US)

(72) Inventor: Mark A. SUSSMAN, San Diego, CA (US)

(21) Appl. No.: 14/304,920

(22) Filed: Jun. 14, 2014

Related U.S. Application Data
(63) Continuation of application No. 13/319,512, filed on Dec. 11, 2011, now abandoned.
(60) Provisional application No. 61/179,578, filed on May 19, 2009.

(51) Int. Cl. A61K 35/39

(52) U.S. Cl. CPC A61K 35/39; A61K 48/00
USPC 424/93.21

(57) ABSTRACT

Disclosed are methods of protecting cells, especially non-vascular system, non-hematopoietic cells and tissues, from apoptosis and enhancing their engraftment, survival, and/or persistence by providing enhanced levels of PIM activity for the cell, including PIM-1 activity. Also disclosed are cells that have been engineered to express enhanced levels of PIM kinase, and methods of administering those cells to vertebrates.

### CROSS-REFERENCE TO RELATED APPLICATIONS

This United States utility patent application is a continuation of U.S. patent application Ser. No. 13/319,512, filed Nov. 8, 2011 (now pending), which is a §371 national phase of PCT international patent application no PCT/SG2010/000147, having an international filing date of May 18, 2010, which claims benefit of priority to U.S. Provisional Patent Application Ser. No. 61/179,578, filed May 19, 2009. The aforementioned applications are expressly incorporated herein by reference in their entirety and for all purposes.

### TECHNICAL FIELD

This invention generally relates to cell and molecular biology and regenerative medicine. This disclosure relates to enhancement of cellular function and survival, including engraftment and persistence of implanted cells or tissues by increasing their exposure to a PIM serine/threonine kinase, including (but limited to) PIM-1, PIM-1, and PIM-3.

### BACKGROUND OF THE INVENTION

PIM-1 is a serine/threonine kinase originally discovered as the proviral integration site for Moloney Murine Leukemia Virus. It was originally believed to function primarily in the hematopoietic system, where it was demonstrated to upregulate hematopoiesis and to facilitate cell growth. Recently, overexpression of PIM-1 was found to protect the myocardium following infarction injury, and to protect cardiomyocytes from apoptotic challenge by increasing cell-survival signaling.

Although PIM-1 has been extensively studied in connection with its proto-oncogenic properties and its effects on the hematopoietic system, and more recently in connection with its role in cardioprotection and cardiac muscle repair, it has not previously been known to have any beneficial or desired properties in other cell types and other tissues.

PIM-1 exists in two isoforms with molecular weights of 34 and 44 kDa. The 34 kDa isoform is cytosolic and nuclear localized, while the 44 kDa isoform was recently found to be membrane bound. PIM-1 may be a relatively promiscuous kinase based upon minimal target substrate recognition sequence requirements and capacity for autophosphorylation.

Induction of PIM-1 expression is mediated by cytokines and growth factors including LIF, GM-CSF, EGF, and most interleukins, consistent with a role for PIM-1 in proliferation and survival of hematopoietic cells. PIM-1 mediates proliferative actions through phosphorylation of multiple target substrates, resulting in cell cycle transition, as well as protective effects via phosphorylation of multiple targets. Induction of PIM-1 expression has been linked to AKT (a serine/threonine kinase) in hematopoietic cells.

### SUMMARY OF THE INVENTION

One aspect of this disclosure discloses a new role for PIM kinases, including PIM-1, in several other tissue types, where it is useful in facilitating one or more of cell growth, cell survival, engraftment of transplanted cells, and persistence of transplanted cells while maintaining function.

One aspect of this disclosure is increasing the levels of PIM kinase in non-cardiac, non-hematopoietic cells or tissues, thereby providing one or more benefits which may include cytoprotection; reduction or reversal of cellular apoptosis; enhanced engraftment or adoptive transfer of cells into a tissue; enhanced survival of engrafted cells; persistence of engrafted cells; enhanced proliferation of stem cells or progenitor cells; and maintenance of function by those cells long after their introduction.

Cell or tissue types of particular interest include pancreatic tissue cells, including islet or beta cells; nervous system tissues, including central and peripheral neurons and glial cells; muscle cells, including non-vascular smooth muscle cells, including cells of gastrointestinal origin; hepatocytes; renal tissue cells, including parenchymal and stromal cells; skeletal cells, including osteoblasts, osteoclasts, and osteocytes; connective tissue cells, including chondroblasts and chondrocytes; any endocrine or hormone-secreting cell, including thyroid, parathyroid, pituitary, and adrenal cells; and pulmonary tissue cells, including pneumocytes. Also included are stem cells and progenitor cells for these various tissues and cells.

For any of these tissue and cell types, levels of PIM kinase can be increased by local expression or exogenous introduction. Local expression can result from induction and expression of endogenously-encoded PIM kinase, introduction of PIM kinase protein, or introduction of exogenous polynucleotide encoding a PIM kinase.

Engineered cells of each of the foregoing types into which polynucleotide encoding PIM-1 has been introduced are specifically contemplated. The polynucleotide can include DNA or RNA.

Methods of transforming cells, implanting cells or tissues, preventing or retarding death of endogenous or transplanted tissues, preventing or reducing cell damage upon contact with a cytotoxic agent or event, and treating or preventing disease or damage of cells or tissues from hypoxia, ischemia, trauma, chemical insult, autoimmune attack, and unwanted apoptosis by introducing or expressing PIM are also expressly contemplated.

One disclosed embodiment is a method, comprising providing an enhanced level of a PIM kinase in a targeted population of non-vascular, non-hematopoietic cells in vivo. The enhanced level can be provided, for example, by delivering an exogenous PIM kinase to the cell population or by causing enhanced production of the PIM kinase by the cell population. In some embodiments, the cell population has been engineered in vivo, in vitro, or ex vivo to include an exogenous polynucleotide sequence operably encoding (operably linked to) the PIM kinase. In alternative embodiments, advantageously the cell population comprises stem cells or progenitor cells, or is an endogenous cell population. In some embodiments, the PIM kinase is PIM-1, PIM-2, or PIM-3. Various cell populations can be used or targeted, such as a neural cell population, a pancreatic cell population such as a pancreatic islet cell population or other pancreatic cells, or any insulin-secreting cell population. The cells may also be an endocrine cell population, a bone cell population, a connective tissue cell population, a renal cell population, a hepatic cell population, or a pulmonary cell population, or a progenitor of any of the foregoing, to name a few examples. The method can further include administering the engineered cells to a mammal, such as a human, or to any vertebrate.

Another aspect relates to a population of non-vascular system, non-hematopoietic cells that has been engineered to express enhanced levels of a PIM kinase. The cell population can comprise stem cells or progenitor cells, for example. In some embodiments, the PIM kinase is PIM-1. Various cell populations can be used, such as a neural cell population, a pancreatic cell population such as a pancreatic islet cell population or other pancreatic cells, or any insulin-secreting cell population. The cells may also be an endocrine cell population, a bone cell population, a connective tissue cell population, a renal cell population, a hepatic cell population, or a pulmonary cell population, to name a few examples.

Also disclosed is a recombinant polynucleotide, comprising a first region encoding a PIM kinase, and a tissue-specific promoter operably linked to the first region, wherein the promoter is specific for a tissue other than a vascular system tissue or a hematopoietic system tissue. In various embodiments, the promoter is specific for a hepatic tissue, a renal tissue, a connective tissue, an endocrine tissue, a bone tissue, a pulmonary tissue, a pancreatic tissue, or a neural tissue.

In alternative embodiments the disclosure provides methods comprising identifying a patient suffering from or at risk of a non-cardiac ischemic condition, a renal disorder, a hepatic disorder, a neural disorder, a connective tissue disorder, an endocrine disorder, a pancreatic disorder, a bone disorder, or a pulmonary disorder; and enhancing levels of PIM kinase at an actual or potential site of the condition or disorder to facilitate cellular survival, proliferation, implantation, or persistence. In various embodiments, PIM kinase levels are enhanced by administering exogenous PIM kinase to the patient, or by administering cells to the patient that express enhanced levels of PIM kinase. Advantageous types of cells include the various tissue types discussed above, and may include progenitor cells or stem cells, as well as autologous cells.

In alternative embodiments the disclosure provides materials comprising PIM kinase or a recombinant polynucleotide encoding PIM kinase for use in increasing PIM kinase levels in a non-vascular, non-cardiac, non-hematopoietic cell population in vivo, thereby enhancing cellular proliferation, survival, implantation, or persistence in that cell population. The cell population can be a neural cell population, a pancreatic cell population, an endocrine cell population, a bone cell population, a renal cell population, a connective tissue cell population, a hepatic cell population, or a pulmonary cell population; or the cell population can include progenitor cells or stem cells.

In alternative embodiments, the materials are (comprise) a recombinant DNA under the control of a promoter. In alternative embodiments, the materials further comprise a host cell containing said recombinant DNA in a manner that the recombinant DNA is expressed in the host cell.

In alternative embodiments, the host cell is a progenitor cell for said cell population, for use in transplantation into a mammal, including a human; or the host cell is a homologous cell of said mammal that has been transformed with said recombinant DNA prior to said transplantation.

In alternative embodiments, the invention provides uses of a material comprising a PIM kinase or a recombinant polynucleotide encoding PIM kinase for the manufacture of a medicament for increasing PIM kinase levels in a non-vascular, non-cardiac, non-hematopoietic cell population in vivo thereby enhancing cellular proliferation, survival, implantation, or persistence in that cell population.

All publications, patents, patent applications, GenBank sequences and ATCC deposits, cited herein are hereby expressly incorporated by reference for all purposes.

### DETAILED DESCRIPTION

In alternative embodiments, the invention provides methods and compositions that provide an enhanced level of a PIM kinase in a targeted population of non-vascular, non-hematopoietic cells in vivo. In one embodiment, the enhanced level is provided by delivering an exogenous PIM kinase to the cell population.

PIM-1 exists in two isoforms with molecular weights of 34 and 44 kDa; the 34 kDa isoform is cytosolic and nuclear localized, while the 44 kDa isoform is membrane bound. PIM-1 may be a relatively promiscuous kinase. Two additional family members, PIM-2 and PIM-3, may exhibit functional redundancy with PIM-1, and in the present disclosure, can be substituted to the extent of that redundancy or based on other inherent function of those members.

We have recognized that the role of PIM-1 is not as limited as was previously believed. Various other cell types can be affected by this kinase to achieve physiologically-desirable results. Such results may include survival of transplanted tissue; survival of transplanted cells; protection from insult, including ischemic insults, cytokine insult, and insult from external factors or cytotoxic agents; facilitation of growth, integration or implantation, and persistence of transplanted or implanted tissues or cells (while maintaining function). Other PIM kinases, including the various isoforms, can similarly be used.

One of the attractive properties of progenitor cells that over-express a PIM kinase is that they undergo asymmetric division, providing one differentiated cell of the particular tissue in question, and one progenitor cell that will undergo further asymmetric division.

In alternative embodiments, the term “PIM” is used herein to refer to a serine or threonine kinase, having PIM activity, including the various PIM enzymes, e.g., PIM-1, PIM-2, and PIM-3, further including any isoforms thereof. For example, the serine/threonine kinase PIM-1 is known to exist in two isoforms, and references to PIM and PIM-1 herein are intended to encompass both isoforms, unless otherwise specified. In addition, although certain cells, constructs, polynucleotides, techniques, uses, and methods are described in connection with one particular PIM, such as PIM-1, such descriptions are exemplary, and should be taken as also including the other PIM enzymes having similar activity.

The term “PIM activity” and “PIM kinase activity” refer to the enzymatic or physiological activity of the PIM enzymes, e.g., the activity of a PIM-1, and encompasses use of other materials having similar activity. The discoveries set forth herein relate to altering characteristics of living cells by enhancing a particular kinase activity in the cells. Of course, as is well known, enzyme variants exist or can be readily constructed, having conservative amino acid substitutions, cross-linking, cross-species domain substitutions, truncations, and the like, while preserving a physiologically-effective level of enzymatic activity (in this case, kinase activity for the PIM-1 target). The present discoveries are not focused only on a particular kinase, but include the discovery of an entirely new role for PIM kinase activity in vascular system cells and tissues. Thus, the results discussed herein flow from alteration of PIM kinase activity, regardless of the exact modality by which that is achieved.

The term “stem cell” is used broadly to include totipotent, pluripotent, and multipotent cells that can differentiate into vascular system cells, including cardiac cells. “Progenitor cells” overlaps somewhat with multipotent stem cells, and includes cells that are at least partially differentiated but that are multipotent or unipotent, in that they have the ability to differentiate into at least one type of mature cell. Various stem cells can be used, including those derived from embryonic stem cells, as well as adult or somatic stem cells; e.g., peripheral stem cells, bone stem cells, neural stem cells, mesenchymal stem cells, adipose-derived stem cells, endothelial stem cells, and the like.

The terms “treat” and “treatment” are used broadly, to include both prophylactic and therapeutic treatments. Similarly, when referring to disease or injury of circulatory system tissues, those terms are used broadly to include fully developed disease or injury, as well as incipient or threatened disease or injury. Thus, a patient at risk of or beginning to develop a particular condition, is considered to have that condition “treated” when methods as disclosed herein are used to reduce the risk of development or progression of that condition, as well as when an already-developed condition is reversed, inhibited, cured, or ameliorated, and when the rate of development of a condition is halted or slowed.

In alternative embodiments, “Vascular tissue” or “vascular system tissue” means blood vessels and cardiac tissue.

Those being treated are referred to variously as patients, individuals, subjects, humans, or animals. Treatments identified as useful for one category are also useful for other categories, and selection of a particular term (other than “human”) is not intended to be limiting, but rather just a use of an alternative expression.

The disclosure includes compositions, such as pharmaceutical compositions, comprising nucleic acids encoding a PIM serine/threonine kinase, such as PIM-1, and methods for making and using them; including methods for inducing cellular proliferation, and protecting particular cells or tissues from hypoxia and cellular apoptosis.

Also disclosed are compositions, such as pharmaceutical compositions, comprising nucleic acids encoding the serine/threonine kinase PIM-1 or other PIM kinases, and methods for preventing or inhibiting cell or tissue damage, e.g., cardiomyocyte cell death or inhibiting an ischemic or reperfusion related injury; including preventing or inhibiting the irreversible cellular and tissue damage and cell death caused by ischemia, e.g., ischemia subsequent to reperfusion (which can exacerbates ischemic damage by activating inflammatory response and oxidative stress).

The disclosure further provides compositions, such as pharmaceutical compositions, comprising PIM proteins (i.e., a kinase having PIM activity) or nucleic acids encoding a serine/threonine kinase PIM.

### PIM Sequences

One aspect of the disclosure includes introduction of an exogenous PIM construct into cells, tissues, or whole organisms. Some embodiments utilize nucleic acid constructs comprising a PIM-encoding sequence, e.g., a PIM-1 expressing message or a PIM-1 gene. In one aspect, PIM-expressing nucleic acids used to practice this invention include PIM-1 genomic sequences, or fragments thereof, including coding or non-coding sequences, e.g., including introns, 5′ or 3′ non-coding sequences, and the like. Also encompassed are PIM-encoding mRNA sequences.

In one aspect, the PIM-1 expressing, or PIM-1 inducing or upregulating, composition is a nucleic acid, including a vector, recombinant virus, and the like; and a recombinant PIM-1 is expressed in a cell in vitro, ex vivo and/or in vivo.

In one aspect, a PIM-1 expressing nucleic acid, e.g., an expression vector, used to practice this invention encodes a human PIM-1, such as GenBank accession no. AAA36447 (see also, e.g., Domen (1987) Oncogene Res. 1 (1):103-112), SEQ ID NO:1.

In another aspect, a PIM-1 expressing nucleic acid, e.g., an expression vector, used to practice this invention encodes a human PIM-1 kinase 44 kDa isoform, see e.g., GenBank accession no. AAY87461 (see also, e.g., Xie (2006) Oncogene 25 (1), 70-78), SEQ ID NO:2.

In a further aspect, a PIM-1 expressing nucleic acid, e.g., an expression vector, used to practice this invention comprises a human PIM-1 kinase message (mRNA), see e.g., GenBank accession no. NM_{—}002648 (see also, e.g., Zhang (2007) Mol. Cancer Res. 5 (9), 909-922), SEQ ID NO:3.

Also disclosed are human DNA sequences of PIM-2 (SEQ ID NO:4) and PIM-3 (SEQ ID NO:8). In a further aspect, the genomic sequence PIM-2 (SEQ ID NO:4) and/or the CDS (or protein coding sequence therein, e.g., SEQ ID NO:5); and/or the genomic sequence PIM-3 (SEQ ID NO:8) and/or the CDS (or protein coding sequence therein, e.g., SEQ ID NO:9); are used to practice this invention and are contained in a PIM-1 expressing nucleic acid, e.g., an expression vector.

In alternative embodiments, nucleic acids of this invention are operatively linked to a transcriptional regulatory sequence, e.g., a promoter and/or an enhancer, e.g., tissue-specific, promoters to drive (e.g., regulate) expression of PIM-1. Promoters and enhancers used to practice this invention can be of any type and/or origin, an in one embodiment promoters specific to the species receiving the PIM-1 construct are used; e.g., humans can receive human promoters, mice receive murine promoters, etc. In other embodiments, promoters from heterologous species can be used; e.g., mammals or vertebrates receiving promoters that originate from other mammals or vertebrates, or viral or synthetic promoters active in the appropriate species and/or cell type also can be used. These promoters can comprise, for example, neuron-specific promoters such as aex-3, che-3, daf-19, eat-4, eat-16, and ehs-1; pancreatic specific promoters such as the pancreatic glucokinase promoter, SEL1L, KLKS and KLK7; bone specific promoters such as the osteocalcin promoter; and any other promoter that drives expression in the target tissue but does not drive significant expression in other tissues. In one embodiment, promoters and enhancers active in primordial cells or stem cells, e.g., neural stem cells, endothelial stem cells, and the like, can be operatively linked to drive expression of PIM-1.

In addition to nucleic acid-driven strategies, PIM protein itself can be directly administered to cells, either in vitro or in vivo. This can be done, for example, by injection, infusion, topical application (e.g., to pulmonary tissue), or through use of protein transduction domains or other protein delivery techniques.

### Nucleic Acid Delivery—Gene Delivery Vehicles

In one aspect, this disclosure provides constructs or expression vehicles, e.g., expression cassettes, vectors, viruses (e.g., lentiviral expression vectors, e.g., see SEQ ID NO:13), and the like, comprising a PIM-encoding sequence, e.g., a PIM-1 encoding message or a PIM-la gene, for use as ex vivo or in vitro gene therapy vehicles, or for expression of PIM-1 in a target cell, tissue or organ to practice the methods of this invention, and for research, drug discovery or transplantation.

In one aspect, an expression vehicle used to practice the invention can comprise a promoter operably linked to a nucleic acid encoding a PIM protein (or functional subsequence thereof). For example, the invention provides expression cassettes comprising nucleic acid encoding a PIM-1 protein operably linked to a transcriptional regulatory element, e.g., a promoter.

In one aspect, an expression vehicle used to practice the invention is designed to deliver a PIM-1 encoding sequence, e.g., a PIM-1 gene or any functional portion thereof to a tissue or cell of an individual. Expression vehicles, e.g., vectors, used to practice the invention can be non-viral or viral vectors or combinations thereof. The invention can use any viral vector or viral delivery system known in the art, e.g., adenoviral vectors, adeno-associated viral (AAV) vectors, herpes viral vectors (e.g., herpes simplex virus (HSV)-based vectors), retroviral vectors, and lentiviral vectors.

In one aspect of the invention, an expression vehicle, e.g., a vector or a virus, is capable of accommodating a full-length PIM-1 gene or a message, e.g., a cDNA. In one aspect, the invention provides a retroviral, e.g., a lentiviral, vector capable of delivering the nucleotide sequence encoding full-length human PIM-1 in vitro, ex vivo and/or in vivo. An exemplary lentiviral expression vector backbone (no “payload” included, e.g., no PIM-1 sequence included) that can be used to practice this invention is set forth in SEQ ID NO:13.

In one embodiment, a lentiviral vector used to practice this invention is a “minimal” lentiviral production system lacking one or more viral accessory (or auxiliary) gene. Exemplary lentiviral vectors for use in the invention can have enhanced safety profiles in that they are replication defective and self-inactivating (SIN) lentiviral vectors. Lentiviral vectors and production systems that can be used to practice this invention include e.g., those described in U.S. Pat. Nos. 6,277,633; 6,312,682; 6,312,683; 6,521,457; 6,669,936; 6,924,123; 7,056,699; and 7,198,784; any combination of these are exemplary vectors that can be employed in the practice of the invention. In an alternative embodiment, non-integrating lentiviral vectors can be employed in the practice of the invention. For example, non-integrating lentiviral vectors and production systems that can be employed in the practice of the invention include those described in U.S. Pat. No. 6,808,923.

The expression vehicle can be designed from any vehicle known in the art, e.g., a recombinant adeno-associated viral vector as described, e.g., in U.S. Pat. App. Pub. No. 20020194630, Manning, et al.; or a lentiviral gene therapy vector, e.g., as described by e.g., Dull, et al. (1998) J. Virol. 72:8463-8471; or a viral vector particle, e.g., a modified retrovirus having a modified proviral RNA genome, as described, e.g., in U.S. Pat. App. Pub. No. 20030003582; or an adeno-associated viral vector as described e.g., in U.S. Pat. No. 6,943,153, describing recombinant adeno-associated viral vectors for use in the eye; or a retroviral or a lentiviral vector as described in U.S. Pat. Nos. 7,198,950; 7,160,727; 7,122,181 (describing using a retrovirus to inhibit intraocular neovascularization in an individual having an age-related macular degeneration); or U.S. Pat. No. 6,555,107.

Any viral vector can be used to practice this invention, and the concept of using viral vectors for gene therapy is well known; see e.g., Verma and Somia (1997) Nature 389:239-242; and Coffin et al (“Retroviruses” 1997 Cold Spring Harbour Laboratory Press Eds: J M Coffin, S M Hughes, H E Varmus pp 758-763) having a detailed list of retroviruses. Any lentiviruses belonging to the retrovirus family can be used for infecting both dividing and non-dividing cells with a PIM-1-encoding nucleic acid, see e.g., Lewis et al (1992) EMBO J. 3053-3058.

Viruses from lentivirus groups from “primate” and/or “non-primate” can be used; e.g., any primate lentivirus can be used, including the human immunodeficiency virus (HIV), the causative agent of human acquired immunodeficiency syndrome (AIDS), and the simian immunodeficiency virus (SIV); or a non-primate lentiviral group member, e.g., including “slow viruses” such as a visna/maedi virus (VMV), as well as the related caprine arthritis-encephalitis virus (CAEV), equine infectious anemia virus (EIAV) and/or a feline immunodeficiency virus (FIV) or a bovine immunodeficiency virus (BIV).

In alternative embodiments, lentiviral vectors used to practice this invention are pseudotyped lentiviral vectors. In one aspect, pseudotyping used to practice this invention incorporates in at least a part of, or substituting a part of, or replacing all of, an env gene of a viral genome with a heterologous env gene, for example an env gene from another virus. In alternative embodiments, the lentiviral vector of the invention is pseudotyped with VSV-G. In an alternative embodiment, the lentiviral vector of the invention is pseudotyped with Rabies-G.

Lentiviral vectors used to practice this invention may be codon optimized for enhanced safety purposes. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms. Codon optimization has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. Codon optimization also overcomes the Rev/RRE requirement for export, rendering optimized sequences Rev independent. Codon optimization also reduces homologous recombination between different constructs within the vector system (for example between the regions of overlap in the gag-pol and env open reading frames). The overall effect of codon optimization is therefore a notable increase in viral titer and improved safety. The strategy for codon optimized gag-pol sequences can be used in relation to any retrovirus.

Vectors, recombinant viruses, and other expression systems used to practice this invention can comprise any nucleic acid which can infect, transfect, transiently or permanently transduce a cell. In one aspect, a vector used to practice this invention can be a naked nucleic acid, or a nucleic acid complexed with protein or lipid. In one aspect, a vector used to practice this invention comprises viral or bacterial nucleic acids and/or proteins, and/or membranes (e.g., a cell membrane, a viral lipid envelope, etc.). In one aspect, expression systems used to practice this invention comprise replicons (e.g., RNA replicons, bacteriophages) to which fragments of DNA may be attached and become replicated. In one aspect, expression systems used to practice this invention include, but are not limited to RNA, autonomous self-replicating circular or linear DNA or RNA (e.g., plasmids, viruses, and the like, see, e.g., U.S. Pat. No. 5,217,879), and include both the expression and non-expression plasmids.

In one aspect, a recombinant microorganism or cell culture used to practice this invention can comprise “expression vector” including both (or either) extra-chromosomal circular and/or linear nucleic acid (DNA or RNA) that has been incorporated into the host chromosome(s). In one aspect, where a vector is being maintained by a host cell, the vector may either be stably replicated by the cells during mitosis as an autonomous structure, or is incorporated within the host's genome.

In one aspect, an expression system used to practice this invention can comprise any plasmid, which are commercially available, publicly available on an unrestricted basis, or can be constructed from available plasmids in accord with published procedures. Plasmids that can be used to practice this invention are well known in the art.

In alternative aspects, a vector used to make or practice the invention can be chosen from any number of suitable vectors known to those skilled in the art, including cosmids, YACs (Yeast Artificial Chromosomes), megaYACS, BACs (Bacterial Artificial Chromosomes), PACs (P1 Artificial Chromosome), MACs (Mammalian Artificial Chromosomes), a whole chromosome, or a small whole genome. The vector also can be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook. Bacterial vectors which can be used include commercially available plasmids comprising genetic elements of known cloning vectors.

### Gene Delivery Methods

The PIM-1 expressing nucleic acid compositions of the invention can be delivered for ex vivo or in vivo gene therapy to deliver a PIM-1 encoding nucleic acid. In one aspect, PIM-1 expressing nucleic acid compositions of the invention, including non-reproducing viral constructs expressing high levels of the human PIM-1 protein, are delivered ex vivo or for in vivo gene therapy.

The PIM-1 expressing nucleic acid compositions of the invention can be delivered to and expressed in a variety of cell types to induce cellular proliferation, and/or to protect cells from hypoxia and cellular apoptosis. PIM-1 so expressed (by practicing the composition and methods of this invention) can protect cells from hypertrophy and inhibit cell death induced by ischemic events, traumatic injury, chemical injury, cytokine injury, and the like. In addition, PIM-1 overexpression (by practicing the composition and methods of this invention) results in cellular reversion; the cells can become stem-cell-like; complete with re-expression of stem cell markers.

In one aspect, overexpression of PIM-1 (by practicing the compositions and methods of this invention) enhances the regenerative potential of stem cells and their ability to repair a damaged or injured organ or tissue. In one aspect, the invention provides compositions and methods for overexpressing PIM-1 using a controlled system using cultured stem cells prior to reintroduction in the adult human to enhance their ability to repair the organ following injury.

In some embodiments, PIM-1 can be used for a clinical therapy for repair of a number of tissues damaged by low oxygen or other means through use of a conditional control element that allows control of PIM-1 expression. For example, PIM-1 expressing nucleic acid delivery vehicles, e.g., expression constructs, such as vectors or recombinant viruses, can be injected directly into the organ to protect it from immediate injury. Expression of the protein can be then activated by administering an activator such as a drug; e.g., through action of the drug on an inducer in the expression construct.

In one embodiment, vectors used to practice this invention, e.g., to generate a PIM-expressing cell, are bicistronic. In one embodiment, a MND (or, myeloproliferative sarcoma virus LTR-negative control region deleted) promoter is used to drive Pim-1 expression. In one embodiment, a reporter is also used, e.g., an enhanced green florescent protein (eGFP) reporter, which can be driven off a viral internal ribosomal entry site (vIRES). In alternative embodiments, all constructs are third generation self-inactivating (SIN) lentiviral vectors and incorporate several elements to ensure long-term expression of the transgene. For example, a MND promoter allows for high expression of the transgene, while the LTR allows for long-term expression after repeated passage. In alternative embodiments, the vectors also include (IFN)-β-scaffold attachment region (SAR) element; SAR elements have been shown to be important in keeping the vector transcriptionally active by inhibiting methylation and protecting the transgene from being silenced.

In alternative embodiments, as a secondary course of therapy, PIM-1 expressing nucleic acid delivery vehicles, e.g., expression constructs, such as vectors or recombinant viruses, can be used to enhance proliferation during culture of adult stem cells extracted from the patient's damaged organ or other tissue. In alternative embodiments, blood, fat, bone, neural, mesenchymal, marrow-derived, and other types of stem cells can be used. PIM-1 expression can be activated through addition of the drug to culture media. After a number of days in culture, the expression of PIM-1 can be then turned off through removal of the drug; and, in one aspect, the increased number of cells produced in culture are reintroduced into the damaged area, contributing to an enhanced repair process.

The invention can incorporate use of any non-viral delivery or non-viral vector systems are known in the art, e.g., including lipid mediated transfection, liposomes, immunoliposomes, LIPOFECTIN™ brand cationic lipid transfection agent, cationic facial amphiphiles (CFAs) and combinations thereof. Other DNA or RNA delivery techniques can also be used, such as electroporation, naked DNA techniques, gold particles, gene guns, and the like.

In one aspect, expression vehicles, e.g., vectors or recombinant viruses, used to practice the invention are injected directly into the heart muscle. In one aspect, the PIM-1 encoding nucleic acid is administered to the individual by direct injection. Thus, in one embodiment, the invention provides sterile injectable formulations comprising expression vehicles, e.g., vectors or recombinant viruses, used to practice the invention.

In alternative embodiments, it may be appropriate to administer multiple applications and employ multiple routes, e.g., directly into the tissue and (optionally) also intravenously, to ensure sufficient exposure of target cells (e.g., stem cells or other progenitor cells) to the expression construct. Multiple applications of the expression construct may also be required to achieve the desired effect.

One particular embodiment of the invention is the ex vivo modification of stem cells of any origin or any multipotent cell, pluripotent cell, progenitor cell, or cell of a particular tissue to enhance PIM-1 expression, followed by administration of the modified cells to a human or other mammalian host, or to any vertebrate. The cells may be directly or locally administered, for example, into a target tissue. Alternatively, systemic administration is also contemplated. The stem cells may be autologous stem cells or heterologous stem cells. They may be derived from embryonic sources or from infant or adult organisms. Particular types of stem cells include, but are not limited to, The enhancement of PIM-1 expression may for example be the result of upregulation of the expression of existing chromosomal PIM-1-encoding sequence in the stem cells, or may be the result of insertion of an exogenous polynucleotide operably encoding PIM-1. As discussed in other contexts herein, a PIM-1-encoding insert in such stem cells may advantageously be under inducible expression control. In addition, the use of a “suicide sequence” of known type

In alternative embodiments, one or more “suicide sequences” are also administered, either separately or in conjunction with a nucleic acid construct of this invention, e.g., incorporated within the same nucleic acid construct (such as a vector, recombinant virus, and the like. See, e.g., Marktel S, et al, Immunologic potential of donor lymphocytes expressing a suicide gene for early immune reconstitution after hematopoietic T-cell-depleted stem cell transplantation. Blood 101:1290-1298(2003). Suicide sequences used to practice this invention can be of known type, e.g., sequences to induce apoptosis or otherwise cause cell death, e.g., in one aspect, to induce apoptosis or otherwise cause cell death upon administration of an exogenous trigger compound or exposure to another type of trigger, including but not limited to light or other electromagnetic radiation exposure.

In one aspect, a PIM-encoding nucleic acid-comprising expression construct or vehicle of the invention is formulated at an effective amount of ranging from about 0.05 to 500 μg/kg, or 0.5 to 50 μg/kg body weight, and can be administered in a single dose or in divided doses. However, it should be understood that the amount of a PIM-1 encoding nucleic acid of the invention, or other the active ingredient (e.g., a PIM-1 inducing or upregulating agent) actually administered ought to be determined in light of various relevant factors including the condition to be treated, the age and weight of the individual patient, and the severity of the patient's symptom; and, therefore, the above dose should not be intended to limit the scope of the invention in any way.

In one aspect, a PIM-1 encoding nucleic acid-comprising expression construct or vehicle of the invention is formulated at a titer of about at least 10¹⁰, 10¹¹, 10¹², 10¹³ 10¹⁴, 10¹⁵, 10¹⁶, or 10¹⁷ physical particles per milliliter. In one aspect, the PIM-1 encoding nucleic acid is administered in about 10, 20, 30, 40, 50, 60, 70, 80, 90, 100, 110, 120, 130, 140 or 150 or more microliter (μl) injections. Doses and dosage regimens can be determined by conventional range-finding techniques known to those of ordinary skill in the art. For example, in alternative embodiments, about 10⁶, 10⁷, 10⁸, 10⁹, 10¹⁰, 10¹¹, 10¹², 10¹³, 10¹⁴, 10¹⁵, 10¹⁶ or 10¹⁷ viral (e.g., lentiviral) particles are delivered to the individual (e.g., a human patient) in one or multiple doses.

In other embodiments, a single administration (e.g., a single dose) comprises from about 0.1 μl to 1.0 μl, 10 μl or to about 100 μl of a pharmaceutical composition of the invention. Alternatively, dosage ranges from about 0.5 ng or 1.0 ng to about 10 μg, 100 μg to 1000 μg of PIM-1 expressing nucleic acid is administered (either the amount in an expression construct, or as in one embodiment, naked DNA is injected). Any necessary variations in dosages and routes of administration can be determined by the ordinarily skilled artisan using routine techniques known in the art.

In one embodiment, a PIM-1 expressing nucleic acid is delivered in vivo directly to a heart using a viral stock in the form of an injectable preparation containing pharmaceutically acceptable carrier such as saline. The final titer of the vector in the injectable preparation can be in the range of between about 10⁸ to 10¹⁴, or between about 10¹⁰ to 10¹², viral particles; these ranges can be effective for gene transfer.

In one aspect, PIM-1 expressing nucleic acids (e.g., vector, transgene) constructs are delivered to a target tissue or organ by direct injection, e.g., using a standard percutaneous hypodermic needle, or using catheter based methods under fluoroscopic guidance. Alternatively, PIM-1 expressing nucleic acids (e.g., vector, transgene) constructs are delivered to organs and tissues using a delivery-facilitating moiety, e.g., lipid-mediated gene transfer.

The direct injection or other localized delivery techniques can use an amount of polynucleotide or other vector that is sufficient for the PIM-1 expressing nucleic acids (e.g., vector, transgene) to be expressed to a degree which allows for sufficiently efficacy; e.g., the amount of the PIM-1 expressing nucleic acid (e.g., vector, transgene) injected in a particular tissue or organ can be in the range of between about 10⁸ to 10¹⁴, or between about 10¹⁰ to 10¹², viral particles. The injection can be made deeply into the tissue in a single injection, or be spread throughout the tissue with multiple injections. Where there is a particular area of injury or a defined area otherwise needing treatment, direct injection into that specific area may be desirable. Use of balloon catheters or other vasculature-blocking techniques to retain the polynucleotide or other vector within the area of desired treatment for a length of time can also be used.

In one aspect, the invention combines a therapeutic PIM-1 nucleic acid with a genetic “sensor” that recognizes and responds to the oxygen deprivation that follows reduced blood flow, or ischemia. Such a technique could be used, for example, in treatment or prophylaxis of stroke injury. As soon as the oxygen declines, the sensor turns on the therapeutic gene, thereby protecting the brain or other tissue of interest.

### Direct PIM Delivery

In addition to cellular and nucleic acid approaches, PIM proteins can also be delivered directly to the affected tissues. Because PIM acts intracellularly, it is preferred to utilize a delivery strategy to facilitate intracellular delivery of PIM.

One technique that can be used is to provide the PIM in a vehicle that in taken up by or that fuses with a target cell. Thus, for example, PIM can be encapsulated within a liposome or other vesicle, as described in more detail above in connection with polynucleotide delivery to cells.

Alternatively, the PIM may be linked to a transduction domain, such as TAT protein. In some embodiments, PIM enzyme can be operably linked to a transduction moiety, such as a synthetic or non-synthetic peptide transduction domain (PTD), Cell penetrating peptide (CPP), a cationic polymer, an antibody, a cholesterol or cholesterol derivative, a Vitamin E compound, a tocol, a tocotrienol, a tocopherol, glucose, receptor ligand or the like, to further facilitate the uptake of the PIM by cells.

A number of protein transduction domains/peptides are known in the art and facilitate uptake of heterologous molecules linked to the transduction domains (e.g., cargo molecules). Such peptide transduction domains (PTD's) facilitate uptake through a process referred to as macropinocytosis. Macropinocytosis is a nonselective form of endocytosis that all cells perform.

Exemplary peptide transduction domains (PTD's) are derived from the Drosophila homeoprotein antennapedia transcription protein (AntHD) (Joliot et al., New Biol. 3:1121-34, 1991; Joliot et al., Proc. Natl. Acad. Sci. USA, 88:1864-8, 1991; Le Roux et al., Proc. Natl. Acad. Sci. USA, 90:9120-4, 1993), the herpes simplex virus structural protein VP22 (Elliott and O'Hare, Cell 88:223-33, 1997), the HIV-1 transcriptional activator TAT protein (Green and Loewenstein, Cell 55:1179-1188, 1988; Frankel and Pabo, Cell 55:1189-1193, 1988), and more recently the cationic N-terminal domain of prion proteins. Preferably, the peptide transduction domain increases uptake of the biomolecule to which it is fused in a receptor independent fashion, is capable of transducing a wide range of cell types, and exhibits minimal or no toxicity (Nagahara et al., Nat. Med. 4:1449-52, 1998). Peptide transduction domains have been shown to facilitate uptake of DNA (Abu-Amer, supra), antisense oligonucleotides (Astriab-Fisher et al., Pharm. Res, 19:744-54, 2002), small molecules (Polyakov et al., Bioconjug. Chem. 11:762-71, 2000) and even inorganic 40 nanometer iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug., Chem. 10:186-91, 1999).

Fusion proteins with such trans-cellular delivery proteins can be readily constructed using known molecular biology techniques.

In addition, any of the polynucleotides encoding PIM molecules can be linked to the foregoing domains to facilitate transduction of those polynucleotides into target cells, in vivo or in vitro.

### Methods Using PIM-Enhanced Cells

Many different methods fall within the scope of this disclosure, both literally and those that will be apparent by analogy to those skilled in the art.

For example, with respect to neural tissues, neuronal or glial cells or neural stem cells can be contacted with enhanced levels of PIM in vivo or ex vivo. The technology can be practiced to obtain a prophylactic or therapeutic benefit, and can be practiced with central nervous system cells (e.g., brain and spinal cord) and with peripheral nervous system cells (e.g., motor nerves, sensory nerves). Both neuronal cell populations and glial cell populations can be treated.

In the case of physical injury to nerve cells (including surgery or trauma), one significant concern is apoptosis. Environmental factors often lead to apoptosis of damaged nerve cells, after which regeneration of lost function is difficult or impossible. Thus, in one treatment contemplated herein, PIM-1 or other PIM protein is injected or infused directly to the site of injury. In a preferred embodiment, the PIM protein is coupled to a protein transduction domain, as described above, to facilitate cell entry. This can provide a neuroprotective benefit, reducing the incidence of apoptosis. A cellular repair benefit is also believed to occur, actually promoting the recovery of nerve function. Injection of sufficient protein to achieve a local concentration of between about 0.1 ng/ml and 100 ug/ml is contemplated. Alternatively, local delivery of a PIM-encoding polynucleotide to the site of the injury can be used to provide an anti-apoptotic, neuroprotective, and/or neuro-regenerative benefit.

Other treatments of the nervous system tissue can include treatment of previous injuries where insufficient functional recovery has occurred. Neurons, glial cells, and/or neural stem cells can be transfected with PIM-encoding polynucleotide ex vivo, and then be implanted into the site of injury. Alternatively, PIM-encoding polynucleotide can be administered in vivo to facilitate growth and repair of nervous system tissue.

Glial cells expressing enhanced levels of PIM can be prepared and used to treat demyelination resulting from any number of hereditary or non-hereditary conditions, including phenylketonuria and other aminoacidurias, Tay-Sachs, Niemann-Pick, and Gaucher's diseases, Hurler's syndrome, Krabbe's disease and other leukodystrophies, adrenoleukodystrophies, adrenomyeloneuropathy, Leber's hereditary optic atrophy and related mitochondrial disorders, carbon monoxide toxicity and other syndromes of delayed hypoxic cerebral demyelination, progressive subcortical ischemic demyelination, nutritional deficiencies, Marchiafava-Bignami disease, monophasic disorders such as optic neuritis, acute transverse myelitis, acute disseminated encephalomyelitis, and acute hemorrhagic leukoencephalitis, progressive multifocal leukoencephalopathy, and multiple schlerosis.

Ischemic injury to brain and other central nervous system tissue, including stroke, can lead to apoptosis or other deleterious events. It is contemplated that both PIM protein and PIM-encoding polynucleotide can be administered immediately after a stroke, or even as a prophylactic in the case of a high risk patient.

Autoimmune conditions or chemotoxicity can lead to loss of pancreatic islet cells and their attendant insulin production, resulting in Type 1 diabetes. Enhanced PIM exposure can have a cytoprotective effect, to prevent or delay the complete loss of pancreatic islet cells. Alternatively, a number of approaches using embryonic stem cells, endothelial stem cells, and various other stem cells sources have now succeeded in creating insulin-producing cells. In those cases, transplantation or engraftment of the resulting cells into a patient is highly desirable to ameliorate effects of or even cure diabetes. However, often the conditions that led to loss of islet cells in the first place still persist, whether autoimmune related, cytokine related, or due to other causes. PIM therapy as disclosed herein could be used to enhance both short and long-term survival of such insulin-producing cells. Cells could be transfected with PIM-encoding polynucleotide prior to being introduced into a patient, or PIM protein could be used before and/or after such introduction. The cells themselves could be introduced into the pancreas; into the peritoneal cavity; into the kidney capsule; into the patient in an immune-shielded structure (by coating individual cells or by enclosing them in a larger structure), all as is known in the art.

Cartilage damage and degeneration is a major contributor to health care costs and disability. Research in to regeneration of damaged connective tissue has shown some promise, but is not yet able to fully address some remaining obstacles to widespread use of such techniques. Facilitating implantation and survival of peripheral, mesenchymal, and adipose stem cells that have shown initial promise in restoring function in damaged joints and other connective tissue could provide significant benefits. Administration of PIM proteins, PIM polynucleotides, and/or stem cells or connective tissue cells (e.g., chondroblasts and chondrocytes) that have been altered to express enhanced levels of PIM are all contemplated, using the techniques disclosed in more detail herein.

Bone conditions characterized by osteoporosis or non-healing breaks are also significant conditions for which there are few satisfactory therapies. One treatment option made possible by the present invention is to treat osteoporosis by altering the levels of PIM expression or exposure of osteoblasts, thereby shifting the balance of bone repair in favor of building new bone tissue. In addition, bone progenitor cells or other cells involved in healing of bone tissue could be transfected ex vivo, using techniques further disclosed herein.

### Kits and Libraries

The invention provides kits comprising compositions of this invention and methods of the invention, including PIM-expressing, or PIM-inducing or upregulating compositions and/or nucleic acids of the invention, including vectors, recombinant viruses and the like, transfecting agents, transducing agents, cells and/or cell lines, instructions (regarding the methods of the invention), or any combination thereof. As such, kits, cells, vectors and the like are provided herein.

The invention will be further described with reference to the following examples; however, it is to be understood that the invention is not limited to such examples.

### Example 1

### Preparation of PIM-1 Lentiviral Vectors

A bicistronic lentiviral vector was prepared that is designed to deliver the human Pim-1 gene under control of a myeloproliferative sarcoma virus LTR-negative control region deleted (MND) promoter. The human Pim-1 cDNA was cloned out using primers containing EcoR1 restriction sites at both ends in order to facilitate cloning into the multiple cloning sites within the backbone. Vectors are bicistronic, whereby the MND promoter drives Pim-1 expression and the reporter, eGFP, is driven off a vIRES. All constructs are third generation self-inactivating (SIN) lentiviral vectors and incorporate several elements to ensure long-term expression of the transgene. The MND promoter allows for high expression of the transgene, while the LTR allows for long-term expression after repeated passage; see Miyoshi et al., J. Virol. 72:8150-8157 (1998); Miyoshi et al., Science 283:682-686 (1999). The vectors also include an (IFN)-β-scaffold attachment region (SAR) element. The SAR element has been shown to be important in keeping the vector transcriptionally active by inhibiting methylation and protecting the transgene from being silenced. See, e.g., Agarwal et al., J. Virol. 72:3720-3728 (1998); Auten et al., Hum. Gene Ther. 10: 1389-1399 (1999); Kurre et al., Blood 102:3117-3119 (2003).

Lentiviral constructs were made as described by Swan, et al, Gene Ther. 13:1480-1492 (2006). Briefly, constructs were co-transfected with three packaging plasmids pMDLg/pRRE, pRSV-rev, and vesicular stomatitis virus-G (VSVG) into 293T cells, using calcium phosphate transfection. Media was changed 16 hours later and viral supernatant was harvested 24 and 48 hours later. Concentration (1000×) of the virus using ultracentrifugation allowed production of high titer virus. Concentrated virus was resuspended in serum-free media, frozen in small aliquots and stored at −80° C. for future use. Viral titer was calculated by infecting 293T cells with limiting dilutions of concentrated viral stock overnight. Media was changed in the morning and cells were harvested 48 hours later and analyzed on a FACS machine to determine the percentage of GFP positive.

### Example 2

### Transfection of Neural Stem Cells

Murine neural stem cells are transfected with the lentiviral vector of Example 1 as follows. The stem cells are plated at 0.2×106 in 48-well plates and transduced with lentivirus overnight at an MOI of 10 with 4 ug/ml polybrene. Cells are washed 16 hours later with PBS and fresh media added. Cells are expanded for an additional week and analyzed by flow cytometry to determine the percentage of eGFP positive cells. Transfected stem cells (TSCs) are then grown overnight in STEMLINE neural stem cell expansion medium (Sigma-Aldrich #S3194).

Lv-egfp or Lv-egfp+Pim1 transduced TSCs from 10 cm plates are washed twice with PBS and harvested in 1 ml of Triazol (Invitrogen #15596-026), after which mRNA is obtained as per manufacturer's protocol. cDNA is prepared as per manufacturer's protocol. Apoptosis PCR array (catalog #PAMM-012) and cell proliferation (catalog #APMM-012) both sold under the trademark SUPERARRAY™ (S.A. Biosciences, Qiagen, Germantown, Md.) and run as per manufacturer's protocol.

Uninfected, Lv-egfp, and Lv-egfp+Pim1, TSCs are plated in quadruplicate at 10,000 cells per well in a 24 well plate. Cells are harvested and counted on a hemocytometer. Viable cells are counted by exclusion of trypan blue.

### Example 3

### Transplantation of Neural Stem Cells

Transfected neural stem cells (TSCs) from Example 2 are differentiated into a neuronal lineage using the techniques set forth in U.S. Pat. No. 6,001,654. These cells are then administered to a mouse at the site of a freshly cut peripheral nerve. After 30 days, the tissue is excised, and histological examination reveals implantation and survival of the TSCs.

### Example 4

Insulin-producing cells differentiated from stem cells (see e.g., U.S. Pat. Nos. 7,056,734 and 7,029,915) are electroporated to incorporate an expression vector comprising human PIM-1 (SEQ. ID. NO:1) under the control of a tetracycline inducible promoter. Transfected cells are then selected as in Example 2 and are injected into the kidney capsule of an animal, and expression of PIM-1 is induced in the animal for 30 days. At the end of that time, the cells are observed to have implanted and grown, and are secreting insulin.

### Example 5

### Treatment of Liver Tissue

Liver tissue damaged by alcohol abuse is harvested by biopsy, and healthy hepatocytes are isolated by flow cytometry. These hepatocytes are then transfected with a PIM-1 lentiviral vector comprising PIM-1 linked to the hepato-specific human apoC-II promoter. Transfected cells are selected and expanded in a suitable hepatocyte expansion medium, for example, the medium described in U.S. Pat. No. 7,022,520. Thereafter, the cells are injected back into the liver tissue. Implantation, survival, and persistence of the cells is observed after 60 days.

### Example 6

### Treatment of Kidney Tissue

Renal tissue from a rat with moderate to severe acetaminophen-induced renal damage is obtained by biopsy, and podocytes are isolated and cultivated. These cells are then transfected with an AAV-vector that includes PIM-1 operably linked to a glomerular-specific promoter (see e.g., Wong, et al., Am. J. Physiol. Renal Physiol. 279:F1027-F1032 (2000)). Transfected cells are selected and reintroduced into the kidney by direct injection, and are observed to implant and persist.

### Sequences Useful in Practicing the Invention

The invention provides compositions and methods comprising use of PIM-expressing nucleic acids and PIM polypeptides.

In one embodiment the Human PIM-1 protein is used to practice the compositions and methods of this invention; an exemplary Human PIM-1 protein that can be used is GenBank accession no. AAA36447 (see also, e.g., Domen (1987) Oncogene Res. 1 (1):103-112) (SEQ ID NO:1):

```
(SEQ ID NO: 1)
    1 MLLSKINSLA HLRAAPCNDL HATKLAPGKE KEPLESQYQV GPLLGSGGFG SVYSGIRVSD
  61 NLPVAIKHVE KDRISDWGEL PNGTRVPMEV VLLKKVSSGF SGVIRLLDWF ERPDSFVLIL
121 ERPEPVQDLF DFITERGALQ EELARSFFWQ VLEAVRHCHN CGVLHRDIKD ENILIDLNRG
181 ELKLIDFGSG ALLKDTVYTD FDGTRVYSPP EWIRYHRYHG RSAAVWSLGI LLYDMVCGDI
241 PFEHDEEIIR GQVFFRQRVS SECQHLIRWC LALRPSDRPT FEEIQNHPWM QDVLLPQETA
301 EIHLHSLSPG PSK
```

In one embodiment, a Human PIM-1 protein isoform is used to practice the compositions and methods of this invention; an exemplary Human PIM-1 protein isoform that can be used is the human pim-1 kinase 44 kDa isoform, see e.g., GenBank accession no. AAY87461 (see also, e.g., Xie (2006) Oncogene 25 (1), 70-78) (SEQ ID NO:6):

```
(SEQ ID NO: 2)
    1 mphepheplt ppfsalpdpa gapsrrqsrq rpqlssdsps afrasrshsr natrshshsh
  61 sprhslrhsp gsgscgsssg hrpcadilev gmllskinsl ahlraapcnd lhatklapgk
121 ekeplesqyq vgpllgsggf gsvysgirvs dnlpvaikhv ekdrisdwge lpngtrvpme
181 vvllkkvssg fsgvirlldw ferpdsfvli lerxepvqdl fdfitergal qeelarsffw
241 qvleavrhch ncgvlhrdik denilidlnr gelklidfgs gallkdtvyt dfdgtrvysp
301 pewiryhryh grsaavwslg illydmvcgd ipfehdeeii rgqvffrqrv ssecqhlirw
361 clalrpsdrp tfeeiqnhpw mqdvllpqet aeihlhslsp gpsk
```

In one embodiment, a Human PIM-1 message (mRNA) is used to practice the compositions and methods of this invention; an exemplary Human PIM-1 message that can be used is GenBank accession no. NM 002648 (see also, e.g., Zhang (2007) Mol. Cancer Res. 5 (9), 909-922) (SEQ ID NO:3):

```
(SEQ ID NO: 3)
      1 ccctttactc ctggctgcgg ggcgagccgg gcgtctgctg cagcggccgc ggtggctgag
    61 gaggcccgag aggagtcggt ggcagcggcg gcggcgggac cggcagcagc agcagcagca
  121 gcagcagcag caaccactag cctcctgccc cgcggcgctg ccgcacgagc cccacgagcc
  181 gctcaccccg ccgttctcag cgctgcccga ccccgctggc gcgccctccc gccgccagtc
  241 ccggcagcgc cctcagttgt cctccgactc gccctcggcc ttccgcgcca gccgcagcca
  301 cagccgcaac gccacccgca gccacagcca cagccacagc cccaggcata gccttcggca
  361 cagccccggc tccggctcct gcggcagctc ctctgggcac cgtccctgcg ccgacatcct
  421 ggaggttggg atgctcttgt ccaaaatcaa ctcgcttgcc cacctgcgcg ccgcgccctg
  481 caacgacctg cacgccacca agctggcgcc cggcaaggag aaggagcccc tggagtcgca
  541 gtaccaggtg ggcccgctac tgggcagcgg cggcttcggc tcggtctact caggcatccg
  601 cgtctccgac aacttgccgg tggccatcaa acacgtggag aaggaccgga tttccgactg
  661 gggagagctg cctaatggca ctcgagtgcc catggaagtg gtcctgctga agaaggtgag
  721 ctcgggtttc tccggcgtca ttaggctcct ggactggttc gagaggcccg acagtttcgt
  781 cctgatcctg gagaggcccg agccggtgca agatctcttc gacttcatca cggaaagggg
  841 agccctgcaa gaggagctgg cccgcagctt cttctggcag gtgctggagg ccgtgcggca
  901 ctgccacaac tgcggggtgc tccaccgcga catcaaggac gaaaacatcc ttatcgacct
  961 caatcgcggc gagctcaagc tcatcgactt cgggtcgggg gcgctgctca aggacaccgt
1021 ctacacggac ttcgatggga cccgagtgta tagccctcca gagtggatcc gctaccatcg
1081 ctaccatggc aggtcggcgg cagtctggtc cctggggatc ctgctgtatg atatggtgtg
1141 tggagatatt cctttcgagc atgacgaaga gatcatcagg ggccaggttt tcttcaggca
1201 gagggtctct tcagaatgtc agcatctcat tagatggtgc ttggccctga gaccatcaga
1261 taggccaacc ttcgaagaaa tccagaacca tccatggatg caagatgttc tcctgcccca
1321 ggaaactgct gagatccacc tccacagcct gtcgccgggg cccagcaaat agcagccttt
1381 ctggcaggtc ctcccctctc ttgtcagatg cccgagggag gggaagcttc tgtctccagc
1441 ttcccgagta ccagtgacac gtctcgccaa gcaggacagt gcttgataca ggaacaacat
1501 ttacaactca ttccagatcc caggcccctg gaggctgcct cccaacagtg gggaagagtg
1561 actctccagg ggtcctaggc ctcaactcct cccatagata ctctcttctt ctcataggtg
1621 tccagcattg ctggactctg aaatatcccg ggggtggggg gtgggggtgg gtcagaaccc
1681 tgccatggaa ctgtttcctt catcatgagt tctgctgaat gccgcgatgg gtcaggtagg
1741 ggggaaacag gttgggatgg gataggacta gcaccatttt aagtccctgt cacctcttcc
1801 gactctttct gagtgccttc tgtggggact ccggctgtgc tgggagaaat acttgaactt
1861 gcctctttta cctgctgctt ctccaaaaat ctgcctgggt tttgttccct atttttctct
1921 cctgtcctcc ctcaccccct ccttcatatg aaaggtgcca tggaagaggc tacagggcca
1981 aacgctgagc cacctgccct tttttctgcc tcctttagta aaactccgag tgaactggtc
2041 ttcctttttg gtttttactt aactgtttca aagccaagac ctcacacaca caaaaaatgc
2101 acaaacaatg caatcaacag aaaagctgta aatgtgtgta cagttggcat ggtagtatac
2161 aaaaagattg tagtggatct aatttttaag aaattttgcc tttaagttat tttacctgtt
2221 tttgtttctt gttttgaaag atgcgcattc taacctggag gtcaatgtta tgtatttatt
2281 tatttattta tttggttccc ttcctattcc aagcttccat agctgctgcc ctagttttct
2341 ttcctccttt cctcctctga cttggggacc ttttggggga gggctgcgac gcttgctctg
2401 tttgtggggt gacgggactc aggcgggaca gtgctgcagc tccctggctt ctgtggggcc
2461 cctcacctac ttacccaggt gggtcccggc tctgtgggtg atggggaggg gcattgctga
2521 ctgtgtatat aggataatta tgaaaagcag ttctggatgg tgtgccttcc agatcctctc
2581 tggggctgtg ttttgagcag caggtagcct gctggtttta tctgagtgaa atactgtaca
2641 ggggaataaa agagatctta tttttttttt tatacttggc gttttttgaa taaaaacctt
2701 ttgtcttaaa aaaaaaaaaa aaaaaaaaaa aaaaaaaaaa aaaaaaaaaa a
```

In one embodiment, a Human PIM-2 gene and/or the protein coded therein is used to practice the compositions and methods of this invention; an exemplary Human PIM-2 gene that can be used is (SEQ ID NO:4) and the protein coded therein, or the CDS (the coding sequence), for this Human PIM-2 gene is SEQ ID NO:5:

```
LOCUS NC_000023 5826 bp DNA linear CON Mar. 3, 2008
DEFINITION Homo sapiens chromosome X, reference assembly, complete sequence.
ACCESSION NC_000023 REGION: complement(48655403 . . . 48661228)
VERSION NC_000023.9 GI:89161218
PROJECT GenomeProject: 168
SOURCE Homo sapiens (human)
ORGANISM Homo sapiens
REFERENCE 1 (bases 1 to 5826)
AUTHORS International Human Genome Sequencing Consortium.
TITLE Finishing the euchromatic sequence of the human genome
JOURNAL Nature 431 (7011), 931-945 (2004)
PUBMED 15496913
(SEQ ID NO: 4)
      1 cgcgcgcggc gaatctcaac gctgcgccgt ctgcgggcgc ttccgggcca ccagtttctc
    61 tgctttccac cctggcgccc cccagccctg gctccccagc tgcgctgccc cgggcgtcca
  121 cgccctgcgg gcttagcggg ttcagtgggc tcaatctgcg cagcgccacc tccatgttga
  181 ccaagcctct acaggggcct cccgcgcccc ccgggacccc cacgccgccg ccaggtgagt
  241 acatcctccc ctactgcaac cagacggggt gggctggaat gatgggttgc agcgcggggg
  301 gagggagtcg tggctgggct cagcacgccg ccaccctgac ttcctcgcct ccgcctgcgt
  361 aggaggcaag gatcgggaag cgttcgaggc cgagtatcga ctcggccccc tcctgggtaa
  421 ggggggcttt ggcaccgtct tcgcaggaca ccgcctcaca gatcgactcc aggtatccgt
  481 catgagggtc ttgggagggt caggtgcgtg tggcgggggc gggggtcctg gccctggaat
  541 gctggttgac cgaggagtga gcctgcagag tgtgtagagg accaggtgtg tgtgtgtgtg
  601 tgtccgtgtc cgtgtccgag gagtgagcct gcagtgtgtg tagagggcca ggtgtgtgtg
  661 cgtgcgcgtg tgtgtgtcgg tctaggaggt tatgggcggg gggggggggc agggggcttc
  721 agattccgga gttccttgac cccggggtcc aggctgtgta tgtgtgggaa agcagggacc
  781 tagatgtgag atttgtggga cttttggagg taggtgtcca gtgtggagtc atgcggacca
  841 ggaccctggt acagagttgg ggtgtcgtag agctaaatag gaagattgtg ggcctggggt
  901 atcaggaaat ctagaactca ggacttggag tgatgagtcc tgatgcctga gaacggagag
  961 cccagggcta aggaaggtgg gagagataaa cttggttccg aggacctgga gggcagggga
1021 gacgccctgg tacgcgttct gtggggtgct gtggttgggg accagaaaga ctagagtgct
1081 ggtagatgga ggaatactgg aggtaggcag aaggtctaga ctgggagggg tctggggatc
1141 acctgctggc ctccttatca cggccttctt ctccaggtgg ccatcaaagt gattccccgg
1201 aatcgtgtgc tgggctggtc ccccttggtg agtaccttcg gagcccttcc taacctacct
1261 actccatcac tgatgtattc acctccttgc ttttccaggg gatgtatgac tccctgggcc
1321 ctgtaacagt gagaatactg ccagtccatt tatactccct tggggtgaca tacagttctg
1381 attcacccca attcccctag agccctggat tctcccctcc aacaaacctt taccatcctt
1441 cctccaaaca ctgctggggg actgcccgca gggcgtgctg gtggggaaca aggggcagag
1501 gtcactggtt gccatggtga tggtggctgc ttctctcttg ccgttataac gctaacggac
1561 atcagggcgg gtctgggcaa gttgtagagt tgggagcgcc ccctggcggg ctctagggga
1621 aactgcgcct gcgcagtcca tgggacccaa agggagaggg tgcgcctgcg caatatcggt
1681 atttttgcat ctcggtgaga aaacgtctgc tgccgtgcaa gtcagcagcc tggccaggag
1741 agggctctac ctcatcccag aaggttgctg ctcgaagtgt acctgcgcag ggcttgggga
1801 ggcagtgggg ggcggatttt gtggccccca gcgtttatac tttttttttt ttggagacac
1861 agtctccctc tgttgcccag gctggagtga ggtgacgcga tctcggctca ctgcaacctc
1921 cgtctcctgg gttcaagtga ttctcctgcc tcagcctccc aagtagctgg gactacagga
1981 gcgcacaacc atgcccggct aatttttgta tttttagtag agacagggtt tcaccatgtt
2041 ggccaggcgg gttttgaact gctgacctca ggtgatccgc ctgcctcggc cactcaaagt
2101 gctgggatta caggcatgag ccaccacgcc cggctgcatt tatgactttt ttttttcctt
2161 gagacggagt ttcgctctgc tgcctgggct ggagtgcagt ggcgtgatct cagctcactg
2221 cagcctccac ctcctgggtt caagcgattc tcctgcctca ggctcctgag tagctggaat
2281 tacaggcacc cgctgccatg cccggctaag ttttacgttt ttagtagaga ccgtgtttca
2341 ccatgttggc caggctggtc tcgaacccct gacctagtga tctgcccgcc ttgggcctcc
2401 caaagtgctg ggattacagg cgtgagccac cgcgcccagc ctctaatttt gtatttttag
2461 tagagacggg gtttctccat gttggtcagg ctggtctcga actcccgacc tcaggtgatc
2521 tgcccgtctc ggcctcccaa agtgctggga ttacaggcgt gagccactgc gcagggccac
2581 atttaggctt tttattggct ggttctaggt gcttggtgat gctgacaaaa cacatgataa
2641 cactaagtcc ttttgtgcta ggtcctttgt aataaatcac tcagctgttt aacaaattag
2701 gtatattgac cacctactat atgacagaca taattctaga cactcagcaa agtattacat
2761 aagtattgag agctcatttt gtgctaggtc cttttttact aattgttttc acctgtttaa
2821 caaatattta ttcagcccta ctctgttagc agccactgtt ctagtgcttc atatacgtcc
2881 gtgaacaaaa caaaccatta cacaataagt gtttattgag tgctaactgc ttgtcagagc
2941 ccatgctatt aagtgctgtc atctgtttaa catttattga tcacctgtgt aaggtactat
3001 tctaatctgg gatatgtcag ggaacaaaac aaaacacata atggtggtgc tgcttctgct
3061 gaaagccttc agttgataac cagatttttc tttgtatttt tgcttgtttg ttttgagaca
3121 gctggagtgc agtggtgtga tcttcactgc aacctctgcc ttcttggctc aagcgaccct
3181 cccacctgag cctcccaagt agctgggact acaggtgcat gccaccaagc ctggctaatt
3241 tttgtgtttg tgccattttg cccaggctga tcttgaactc ttgggctcaa gcaatccacc
3301 cacatcagcc tcccaaagtg ctgggattgc agggatgagc cactgtgcct ggccgaactt
3361 ctttcgttta ttcaaatgtt tattgatcta cgacatgcga gatttgtgca ggctctttgc
3421 tggtttcacc ctctcaatcg ctgtgtgagt ttgtgtcttt agggaaagtg aggcccagga
3481 agggaagtga gttgcttagc gacacactgt caggaaaagg ggccctgagt tgagcttagg
3541 taaaaagcct cagagctgtt gccctgacat ctgtcttttt tctctccctg cttcccaccc
3601 cacctgtgcc cccagtcaga ctcagtcaca tgcccactcg aagtcgcact gctatggaaa
3661 gtgggtgcag gtggtgggca ccctggcgtg atccgcctgc ttgactggtt tgagacacag
3721 gagggcttca tgctggtcct cgagcggcct ttgcccgccc aggatctctt tgactatatc
3781 acagagaagg gcccactggg tgaaggccca agccgctgct tctttggcca agtagtggca
3841 gccatccagc actgccattc ccgtggagtt gtccatcgtg acatcaagga tgagaacatc
3901 ctgatagacc tacgccgtgg ctgtgccaaa ctcattgatt ttggttctgg tgccctgctt
3961 catgatgaac cctacactga ctttgatggt aaggcttctc taaatctccc tggagggatt
4021 gtttttactt gatggccttg tgacctttgg cctccagtgg tggggtgtcc tgtaatcctt
4081 gacccatact gcattatata agatgatcga ttgctaatac tggggattct cagccttgcc
4141 ctctgataaa gtccatcttt taatggtgtg ctaaccttat tctgggctcc tattctggtg
4201 aggggatcct gttaccatcc tgagtattct ttctctggta aggggatcct gttacttttc
4261 agtgctttta ttctgttgag gggactctgt tattttagct gctttttatc tagtgagggg
4321 actctgcttt tatcttgagt gctcttaatt gtggtgaggc catccttcct ggagagtttg
4381 gggttggaga agggcatcat gagattgagt tggtctaacc cctggcttgt gtgcagggac
4441 aagggtgtac agccccccag agtggatctc tcgacaccag taccatgcac tcccggccac
4501 tgtctggtca ctgggcatcc tcctctatga catggtgtgt ggggacattc cctttgagag
4561 ggaccaggag attctggaag ctgagctcca cttcccagcc catgtctccc caggtgaggc
4621 ctcactgacc ccagcccaga agactccatc cttctcaggg accagtaccc cctactgact
4681 gctaatcttc cctctctgct tcttggccta cagactgctg tgccctaatc cgccggtgcc
4741 tggcccccaa accttcttcc cgaccctcac tggaagagat cctgctggac ccctggatgc
4801 aaacaccagc cgaggatgta cccctcaacc cctccaaagg aggccctgcc cctttggcct
4861 ggtccttgct accctaagcc tggcctggcc tggcctggcc cccaatggtc agaagagcca
4921 tcccatggcc atgtcacagg gatagatgga catttgttga cttggtttta caggtcatta
4981 ccagtcatta aagtccagta ttactaaggt aagggattga ggatcagggg ttagaagaca
5041 taaaccaagt ctgcccagtt cccttcccaa tcctacaaag gagccttcct cccagaacct
5101 gtggtccctg attctggagg gggaacttct tgcttctcat tttgctaagg aagtttattt
5161 tggtgaagtt gttcccattc tgagccccgg gactcttatt ctgatgatgt gtcaccccac
5221 attggcacct cctactacca ccacacaaac ttagttcata tgctcttact tgggcaaggg
5281 tgctttcctt ccaatacccc agtagctttt attttagtaa agggaccctt tcccctagcc
5341 tagggtccca tattgggtca agctgcttac ctgcctcagc ccaggattct ttattctggg
5401 ggaggtaatg ccctgttgtt accccaaggc ttcttttttt tttttttttt tttgggtgag
5461 gggaccctac tctgttatcc caagtgctct tattctggtg agaagaacct tacttccata
5521 atttgggaag gaatggaaga tggacaccac cggacaccac cagacactag gatgggatgg
5581 atggtttttt gggggatggg ctaggggaaa taaggcttgc tgtttgttct cctggggcgc
5641 tccctccaac ttttgcagat tcttgcaacc tcctcctgag ccgggattgt ccaattacta
5701 aaatgtaaat aatcacgtat tgtggggagg ggagttccaa gtgtgccctc ctctcttctc
5761 ctgcctggat tatttaaaaa gccatgtgtg gaaacccact atttaataaa agtaatagaa
5821 tcagaa
```

In one embodiment, exemplary Human PIM polypeptides and message that can be used are:

```
Human PIM-3 Fragment
(SEQ ID NO: 6)
MLLSKFGSLAHLCGPGGVDHLPVKILQPAKADKESFEKAYQVGA
Human PIM-3 protein, translation from genomic
(SEQ ID NO: 7)
        1 VLGSGGFGTV YAGSRIADGL PVAVKHVVKE RVTEWGSLGG ATVPLEVVLL RKVGAAGGAR
      61 GVIRLLDWFE RPDGFLLVLE RPEPAQDLFD FITERGALDE PLARRFFAQV LAAVRHCHSC
    121 GVVHRDIKDE NLLVDLRSGE LKLIDFGSGA LLKDTVYTDF DGTRVYSPPE WIRYHRYHGR
    181 SATVWSLGVL LYDMVCGDIP FEQDEEILRG RLLFRRRVSP ECQQLIRWCL SLRPSERPSL
    241 DQIAAHPWML GADGGAPESC DLRLCTLDPD DVASTTSSSE SL
Human PIM-3 mRNA,
LOCUS NM_001001852 2392 bp mRNA linear PRI Oct. 22, 2008
DEFINITION Homo sapiens pim-3 oncogene (PIM3), mRNA.
ACCESSION NM_001001852 XM_497821
VERSION NM_001001852.3 GI:52138581
SOURCE Homo sapiens (human)
(SEQ ID NO: 8)
        1 gagagcgtga gcgcggagag cggaccgacg cgacacgccg tgcgcctccg cggctgcgct
      61 acgaaaacga gtcccggagc ggccccgcgc ccgccgcacc cggccctcgc ccgcccgaag
    121 acaggcgcca agctgccccg ccgtctcccc agctagcgcc cggccgccgc cgcctcgcgg
    181 gccccgggcg gaagggggcg gggtcccgat tcgccccgcc cccgcggagg gatacgcggc
    241 gccgcggccc aaaacccccg ggcgaggcgg ccggggcggg tgaggcgctc cgcctgctgc
    301 gcgtctacgc ggtccccgcg ggccttccgg gcccactgcg ccgcgcggac cgcctcgggc
    361 tcggacggcc ggtgtccccg gcgcgccgct cgcccggatc ggccgcggct tcggcgcctg
    421 gggctcgggg ctccggggag gccgtcgccc gcgatgctgc tctccaagtt cggctccctg
    481 gcgcacctct gcgggcccgg cggcgtggac cacctcccgg tgaagatcct gcagccagcc
    541 aaggcggaca aggagagctt cgagaaggcg taccaggtgg gcgccgtgct gggtagcggc
    601 ggcttcggca cggtctacgc gggtagccgc atcgccgacg ggctcccggt ggctgtgaag
    661 cacgtggtga aggagcgggt gaccgagtgg ggcagcctgg gcggcgcgac cgtgcccctg
    721 gaggtggtgc tgctgcgcaa ggtgggcgcg gcgggcggcg cgcgcggcgt catccgcctg
    781 ctggactggt tcgagcggcc cgacggcttc ctgctggtgc tggagcggcc cgagccggcg
    841 caggacctct tcgactttat cacggagcgc ggcgccctgg acgagccgct ggcgcgccgc
    901 ttcttcgcgc aggtgctggc cgccgtgcgc cactgccaca gctgcggggt cgtgcaccgc
    961 gacattaagg acgaaaatct gcttgtggac ctgcgctccg gagagctcaa gctcatcgac
  1021 ttcggttcgg gtgcgctgct caaggacacg gtctacaccg acttcgacgg cacccgagtg
  1081 tacagccccc cggagtggat ccgctaccac cgctaccacg ggcgctcggc caccgtgtgg
  1141 tcgctgggcg tgcttctcta cgatatggtg tgtggggaca tccccttcga gcaggacgag
  1201 gagatcctcc gaggccgcct gctcttccgg aggagggtct ctccagagtg ccagcagctg
  1261 atccggtggt gcctgtccct gcggccctca gagcggccgt cgctggatca gattgcggcc
  1321 catccctgga tgctgggggc tgacgggggc gtcccggaga gctgtgacct gcggctgtgc
  1381 accctcgacc ctgatgacgt ggccagcacc acgtccagca gcgagagctt gtgaggagct
  1441 gcacctgact gggagctagg ggaccacctg ccttggccag acctgggacg cccccagacc
  1501 ctgactttct cctgcgtggg ccgtctcctc ctgcggaagc agtgacctct gacccctggt
  1561 gaccttcgct ttgagtgcct tttgaacgct ggtcccgcgg gacttggttt tctcaagctc
  1621 tgtctgtcca aagacgctcc ggtcgaggtc ccgcctgccc tgggtggata cttgaacccc
  1681 agacgcccct ctgtgctgct gtgtccggag gcggccttcc catctgcctg cccacccgga
  1741 gctctttccg ccggcgcagg gtcccaagcc cacctcccgc cctcagtcct gcggtgtgcg
  1801 tctgggcacg tcctgcacac acaatgcaag tcctggcctc cgcgcccgcc cgcccacgcg
  1861 agccgtaccc gccgccaact ctgttattta tggtgtgacc ccctggaggt gccctcggcc
  1921 caccggggct atttattgtt taatttattt gttgaggtta tttcctctga gcagtctgcc
  1981 tctcccaagc cccaggggac agtggggagg caggggaggg ggtggctgtg gtccagggac
  2041 cccaggccct gattcctgtg cctggcgtct gtcccggccc cgcctgtcag aagatgaaca
  2101 tgtatagtgg ctaacttaag gggagtgggt gaccctgaca cttccaggca ctgtgcccag
  2161 ggtttgggtt ttaaattatt gactttgtac agtctgcttg tgggctctga aagctggggt
  2221 ggggccagag cctgagcgtt taatttattc agtacctgtg tttgtgtgaa tgcggtgtgt
  2281 gcaggcatcg cagatggggg ttctttcagt tcaaaagtga gatgtctgga gatcatattt
  2341 ttttatacag gtatttcaat taaaatgttt ttgtacataa aaaaaaaaaa aaaaaaaaaa
  2401 aaaaaaaaaa
Human PIM-1
(SEQ ID NO: 10)
        1 agcttcgaat tatgctcttg tccaaaatca actcgcttgc ccacctgcgc gccgcgccct
      61 gcaacgacct gcacgccacc aagctggcgc ccggcaagga gaaggagccc ctggagtcgc
    121 agtaccaggt gggcccgcta ctgggcagcg gcggcttcgg ctcggtctac tcaggcatcc
    181 gcgtctccga caacttgccg gtggccatca aacacgtgga gaaggaccgg atttccgact
    241 ggggagagct gcctaatggc actcgagtgc ccatggaagt ggtcctgctg aagaaggtga
    301 gctcgggttt ctccggcgtc attaggctcc tggactggtt cgagaggccc gacagtttcg
    361 tcctgatcct ggagaggccc gagccggtgc aagatctctt cgacttcatc acggaaaggg
    421 gagccctgca agaggagctg gcccgcagct tcttctggca ggtgctggag gccgtgcggc
    481 actgccacaa ctgcggggtg ctccaccgcg acatcaagga cgaaaacatc cttatcgacc
    541 tcaatcgcgg cgagctcaag ctcatcgact tcgggtcggg ggcgctgctc aaggacaccg
    601 tctacacgga cttcgatggg acccgagtgt atagccctcc agagtggatc cgctaccatc
    661 gctaccatgg caggtcggcg gcagtctggt ccctggggat cctgctgtat gatatggtgt
    721 gtggagatat tcctttcgag catgacgaag agatcatcag gggccaggtt ttcttcaggc
    781 agagggtctc ttcagaatgt cagcatctca ttagatggtg cttggccctg agaccatcag
    841 ataggccaac cttcgaagaa atccagaacc atccatggat gcaagatgtt ctcctgcccc
    901 aggaaactgc tgagatccac ctccacagcc tgtcgccggg gcccagcagc ctgtcgccgg
    961 ggcccagcaa acaattggta ccgcgggccc gg
Human PIM-1
(SEQ ID NO: 11)
                  atgctct tgtccaaaat caactcgctt gcccacctgc gcgccgcgcc ctgcaacgac
    421 ctgcacgcca ccaagctggc gcccggcaag gagaaggagc ccctggagtc gcagtaccag
    481 gtgggcccgc tactgggcag cggcggcttc ggctcggtct actcaggcat ccgcgtctcc
    541 gacaacttgc cggtggccat caaacacgtg gagaaggacc ggatttccga ctggggagag
    601 ctgcctaatg gcactcgagt gcccatggaa gtggtcctgc tgaagaaggt gagctcgggt
    661 ttctccggcg tcattaggct cctggactgg ttcgagaggc ccgacagttt cgtcctgatc
    721 ctggagaggc ccgagccggt gcaagatctc ttcgacttca tcacggaaag gggagccctg
    781 caagaggagc tggcccgcag cttcttctgg caggtgctgg aggccgtgcg gcactgccac
    841 aactgcgggg tgctccaccg cgacatcaag gacgaaaaca tccttatcga cctcaatcgc
    901 ggcgagctca agctcatcga cttcgggtcg ggggcgctgc tcaaggacac cgtctacacg
    961 gacttcgatg ggacccgagt gtatagccct ccagagtgga tccgctacca tcgctaccat
  1021 ggcaggtcgg cggcagtctg gtccctgggg atcctgctgt atgatatggt gtgtggagat
  1081 attcctttcg agcatgacga agagatcatc aggggccagg ttttcttcag gcagagggtc
  1141 tcttcagaat gtcagcatct cattagatgg tgcttggccc tgagaccatc agataggcca
  1201 accttcgaag aaatccagaa ccatccatgg atgcaagatg ttctcctgcc ccaggaaact
  1261 gctgagatcc acctccacag cctgtcgccg gggcccagca aatag
Murine PIM-1
(SEQ ID NO: 12)
    100 a tgctcctgtc caagatcaac
    121 tccctggccc acctgcgcgc cgcgccctgc aacgacctgc acgccaccaa gctggcgccg
    181 ggcaaagaga aggagcccct ggagtcgcag taccaggtgg gcccgctgtt gggcagcggt
    241 ggcttcggct cggtctactc tggcatccgc gtcgccgaca acttgccggt ggccattaag
    301 cacgtggaga aggaccggat ttccgattgg ggagaactgc ccaatggcac ccgagtgccc
    361 atggaagtgg tcctgttgaa gaaggtgagc tcggacttct cgggcgtcat tagacttctg
    421 gactggttcg agaggcccga tagtttcgtg ctgatcctgg agaggcccga accggtgcaa
    481 gacctcttcg actttatcac cgaacgagga gccctacagg aggacctggc ccgaggattc
    541 ttctggcagg tgctggaggc cgtgcggcat tgccacaact gcggggttct ccaccgcgac
    601 atcaaggacg agaacatctt aatcgacctg agccgcggcg aaatcaaact catcgacttc
    661 gggtcggggg cgctgctcaa ggacacagtc tacacggact ttgatgggac ccgagtgtac
    721 agtcctccag agtggattcg ctaccatcgc taccacggca ggtcggcagc tgtctggtcc
    781 cttgggatcc tgctctatga catggtctgc ggagatattc cgtttgagca cgatgaagag
    841 atcatcaagg gccaagtgtt cttcaggcaa actgtctctt cagagtgtca gcacc [symbol] tt
    901 aaatggtgcc tgtccctgag accatcagat cggccctcct ttgaagaaat ccggaaccat
    961 ccatggatgc agggtgacct cctgccccag gcagcttctg agatccatct gcacagtctg
  1021 tcaccggggt ccagcaagta g
A lentiviral construct as set forth in Example 1 is disclosed herein as SEQ ID NO: 13:
(SEQ ID NO: 11)
        1 gacggatcgg gagatctccc gatcccctat ggtcgactct cagtacaatc tgctctgatg
      61 ccgcatagtt aagccagtat ctgctccctg cttgtgtgtt ggaggtcgct gagtagtgcg
    121 cgagcaaaat ttaagctaca acaaggcaag gcttgaccga caattgcatg aagaatctgc
    181 ttagggttag gcgttttgcg ctgcttcgcg atgtacgggc cagatatacg cgttgacatt
    241 gattattgac tagttattaa tagtaatcaa ttacggggtc attagttcat agcccatata
    301 tggagttccg cgttacataa cttacggtaa atggcccgcc tggctgaccg cccaacgacc
    361 cccgcccatt gacgtcaata atgacgtatg ttcccatagt aacgccaata gggactttcc
    421 attgacgtca atgggtggag tatttacggt aaactgccca cttggcagta catcaagtgt
    481 atcatatgcc aagtacgccc cctattgacg tcaatgacgg taaatggccc gcctggcatt
    541 atgcccagta catgacctta tgggactttc ctacttggca gtacatctac gtattagtca
    601 tcgctattac catggtgatg cggttttggc agtacatcaa tgggcgtgga tagcggtttg
    661 actcacgggg atttccaagt ctccacccca ttgacgtcaa tgggagtttg ttttggcacc
    721 aaaatcaacg ggactttcca aaatgtcgta acaactccgc cccattgacg caaatgggcg
    781 gtaggcgtgt acggtgggag gtctatataa gcagcgcgtt ttgcctgtac tgggtctctc
    841 tggttagacc agatctgagc ctgggagctc tctggctaac tagggaaccc actgcttaag
    901 cctcaataaa gcttgccttg agtgcttcaa gtagtgtgtg cccgtctgtt gtgtgactct
    961 ggtaactaga gatccctcag acccttttag tcagtgtgga aaatctctag cagtggcgcc
  1021 cgaacaggga cctgaaagcg aaagggaaac cagaggagct ctctcgacgc aggactcggc
  1081 ttgctgaagc gcgcacggca agaggcgagg ggcggcgact ggtgagtacg ccaaaaattt
  1141 tgactagcgg aggctagaag gagagagatg ggtgcgagag cgtcagtatt aagcggggga
  1201 gaattagatc gcgatgggaa aaaattcggt taaggccagg gggaaagaaa aaatataaat
  1261 taaaacatat agtatgggca agcagggagc tagaacgatt cgcagttaat cctggcctgt
  1321 tagaaacatc agaaggctgt agacaaatac tgggacagct acaaccatcc cttcagacag
  1381 gatcagaaga acttagatca ttatataata cagtagcaac cctctattgt gtgcatcaaa
  1441 ggatagagat aaaagacacc aaggaagctt tagacaagat agaggaagag caaaacaaaa
  1501 gtaagaccac cgcacagcaa gcggccgctg atcttcagac ctggaggagg agatatgagg
  1561 gacaattgga gaagtgaatt atataaatat aaagtagtaa aaattgaacc attaggagta
  1621 gcacccacca aggcaaagag aagagtggtg cagagagaaa aaagagcagt gggaatagga
  1681 gctttgttcc ttgggttctt gggagcagca ggaagcacta tgggcgcagc gtcaatgacg
  1741 ctgacggtac aggccagaca attattgtct ggtatagtgc agcagcagaa caatttgctg
  1801 agggctattg aggcgcaaca gcatctgttg caactcacag tctggggcat caagcagctc
  1861 caggcaagaa tcctggctgt ggaaagatac ctaaaggatc aacagctcct ggggatttgg
  1921 ggttgctctg gaaaactcat ttgcaccact gctgtgcctt ggaatgctag ttggagtaat
  1981 aaatctctgg aacagatttg gaatcacacg acctggatgg agtgggacag agaaattaac
  2041 aattacacaa gcttaataca ctccttaatt gaagaatcgc aaaaccagca agaaaagaat
  2101 gaacaagaat tattggaatt agataaatgg gcaagtttgt ggaattggtt taacataaca
  2161 aattggctgt ggtatataaa attattcata atgatagtag gaggcttggt aggtttaaga
  2221 atagtttttg ctgtactttc tatagtgaat agagttaggc agggatattc accattatcg
  2281 tttcagaccc acctcccaac cccgagggga cccgacaggc ccgaaggaat agaagaagaa
  2341 ggtggagaga gagacagaga cagatccatt cgattagtga acggatccga tccacaaatg
  2401 gcagtattca tccacaattt taaaagaaaa ggggggattg gggggtacag tgcaggggaa
  2461 agaatagtag acataatagc aacagacata caaactaaag aattacaaaa acaaattaca
  2521 aaaattcaaa attttcgggt ttattacagg gacagcagag atccagtttg gcctgcagag
  2581 atccagagtt aggcagggac attcaccatt atcgtttcag acccacctcc caaccccggt
  2641 catatgggaa tgaaagaccc cacctgtagg tttggcaagc taggatcaag gttaggaaca
  2701 gagagacagc agaatatggg ccaaacagga tatctgtggt aagcagttcc tgccccggct
  2761 cagggccaag aacagttgga acaggagaat atgggccaaa caggatatct gtggtaagca
  2821 gttcctgccc cggctcaggg ccaagaacag atggtcccca gatgcggtcc cgccctcagc
  2881 agtttctaga gaaccatcag atgtttccag ggtgccccaa ggacctgaaa tgaccctgtg
  2941 ccttatttga actaaccaat cagttcgctt ctcgcttctg ttcgcgcgct tctgctcccc
  3001 gagctctata taagcagagc tcgtttagtg aaccgtcaga tcgcctggag acgccatcca
  3061 cgctgttttg acctccatag aagatcagtt aattaagaat tcgcccctct ccctcccccc
  3121 cccctaacgt tactggccga agccgcttgg aataaggccg gtgtgcgttt gtctatatgt
  3181 tattttccac catattgccg tcttttggca atgtgagggc ccggaaacct ggccctgtct
  3241 tcttgacgag cattcctagg ggtctttccc ctctcgccaa aggaatgcaa ggtctgttga
  3301 atgtcgtgaa ggaagcagtt cctctggaag cttcttgaag acaaacaacg tctgtagcga
  3361 ccctttgcag gcagcggaac cccccacctg gcgacaggtg cctctgcggc caaaagccac
  3421 gtgtataaga tacacctgca aaggcggcac aaccccagtg ccacgttgtg agttggatag
  3481 ttgtggaaag agtcaaatgg ctctcctcaa gcgtattcaa caaggggctg aaggatgccc
  3541 agaaggtacc ccattgtatg ggatctgatc tggggcctcg gtgcacatgc tttacatgtg
  3601 tttagtcgag gttaaaaaaa cgtctaggcc ccccgaacca cggggacgtg gttttccttt
  3661 gaaaaacacg atgataatat ggccacaacc atggtgagca agggcgagga gctgttcacc
  3721 ggggtggtgc ccatcctggt cgagctggac ggcgacgtaa acggccacaa gttcagcgtg
  3781 tccggcgagg gcgagggcga tgccacctac ggcaagctga ccctgaagtt catctgcacc
  3841 accggcaagc tgcccgtgcc ctggcccacc ctcgtgacca ccctgaccta cggcgtgcag
  3901 tgcttcagcc gctaccccga ccacatgaag cagcacgact tcttcaagtc cgccatgccc
  3961 gaaggctacg tccaggagcg caccatcttc ttcaaggacg acggcaacta caagacccgc
  4021 gccgaggtga agttcgaggg cgacaccctg gtgaaccgca tcgagctgaa gggcatcgac
  4081 ttcaaggagg acggcaacat cctggggcac aagctggagt acaactacaa cagccacaac
  4141 gtctatatca tggccgacaa gcagaagaac ggcatcaagg tgaacttcaa gatccgccac
  4201 aacatcgagg acggcagcgt gcagctcgcc gaccactacc agcagaacac ccccatcggc
  4261 gacggccccg tgctgctgcc cgacaaccac tacctgagca cccagtccgc cctgagcaaa
  4321 gaccccaacg agaagcgcga tcacatggtc ctgctggagt tcgtgaccgc cgccgggatc
  4381 actctcggca tggacgagct gtacaagtaa agcggccgca ctgttctcat cacatcatat
  4441 caaggttata taccatcaat attgccacag atgttactta gccttttaat atttctctaa
  4501 tttagtgtat atgcaatgat agttctctga tttctgagat tgagtttctc atgtgtaatg
  4561 attatttaga gtttctcttt catctgttca aatttttgtc tagttttatt ttttactgat
  4621 ttgtaagact tctttttata atctgcatat tacaattctc tttactgggg tgttgcaaat
  4681 attttctgtc attctatggc ctgacttttc ttaatggttt tttaatttta aaaataagtc
  4741 ttaatattca tgcaatctaa ttaacaatct tttctttgtg gttaggactt tgagtcataa
  4801 gaaatttttc tctacactga agtcatgatg gcatgcttct atattatttt ctaaaagatt
  4861 taaagttttg ccttctccat ttagacttat aattcactgg aatttttttg tgtgtatggt
  4921 atgacatatg ggttcccttt tattttttac atataaatat atttccctgt ttttctaaaa
  4981 aagaaaaaga tcatcatttt cccattgtaa aatgccatat ttttttcata ggtcacttac
  5041 atatatcaat gggtctgttt ctgagctcta ctctatttta tcagcctcac tgtctatccc
  5101 cacacatctc atgctttgct ctaaatcttg atatttagtg gaacattctt tcccattttg
  5161 ttctacaaga atatttttgt tattgtcttt gggctttcta tatacatttt gaaatgaggt
  5221 tgacaagttt ctagagttaa ctcgagggat caagcttatc gataatcaac ctctggatta
  5281 caaaatttgt gaaagattga ctggtattct taactatgtt gctcctttta cgctatgtgg
  5341 atacgctgct ttaatgcctt tgtatcatgc tattgcttcc cgtatggctt tcattttctc
  5401 ctccttgtat aaatcctggt tgctgtctct ttatgaggag ttgtggcccg ttgtcaggca
  5461 acgtggcgtg gtgtgcactg tgtttgctga cgcaaccccc actggttggg gcattgccac
  5521 cacctgtcag ctcctttccg ggactttcgc tttccccctc cctattgcca cggcggaact
  5581 catcgccgcc tgccttgccc gctgctggac aggggctcgg ctgttgggca ctgacaattc
  5641 cgtggtgttg tcggggaagc tgacgtcctt tccatggctg ctcgcctgtg ttgccacctg
  5701 gattctgcgc gggacgtcct tctgctacgt cccttcggcc ctcaatccag cggaccttcc
  5761 ttcccgcggc ctgctgccgg ctctgcggcc tcttccgcgt cttcgccttc gccctcagac
  5821 gagtcggatc tccctttggg ccgcctcccc gcatcgatac cgtcgagacc tagaaaaaca
  5881 tggagcaatc acaagtagca acacagcagc taccaatgct gattgtgcct ggctagaagc
  5941 acaagaggag gaggaggtgg gttttccagt cacacctcag gtacctttaa gaccaatgac
  6001 ttacaaggca gctgtagatc ttagccactt tttaaaagaa aaggggggac tggaagggct
  6061 aattcactcc caacgaagac aagatatcct tgatctgtgg atctaccaca cacaaggcta
  6121 cttccctgat tggcagaact acacaccagg gccagggatc agatatccac tgacctttgg
  6181 atggtgctac aagctagtac cagttgagca agagaaggta gaagaagcca atgaaggaga
  6241 gaacacccgc ttgttacacc ctgtgagcct gcatgggatg gatgacccgg agagagaagt
  6301 attagagtgg aggtttgaca gccgcctagc atttcatcac atggcccgag agctgcatcc
  6361 ggactgtact gggtctctct ggttagacca gatctgagcc tgggagctct ctggctaact
  6421 agggaaccca ctgcttaagc ctcaataaag cttgccttga gtgcttcaag tagtgtgtgc
  6481 ccgtctgttg tgtgactctg gtaactagag atccctcaga cccttttagt cagtgtggaa
  6541 aatctctagc agggcccgtt taaacccgct gatcagcctc gactgtgcct tctagttgcc
  6601 agccatctgt tgtttgcccc tcccccgtgc cttccttgac cctggaaggt gccactccca
  6661 ctgtcctttc ctaataaaat gaggaaattg catcgcattg tctgagtagg tgtcattcta
  6721 ttctgggggg tggggtgggg caggacagca agggggagga ttgggaagac aatagcaggc
  6781 atgctgggga tgcggtgggc tctatggctt ctgaggcgga aagaaccagc tggggctcta
  6841 gggggtatcc ccacgcgccc tgtagcggcg cattaagcgc ggcgggtgtg gtggttacgc
  6901 gcagcgtgac cgctacactt gccagcgccc tagcgcccgc tcctttcgct ttcttccctt
  6961 cctttctcgc cacgttcgcc ggctttcccc gtcaagctct aaatcggggc atccctttag
  7021 ggttccgatt tagtgcttta cggcacctcg accccaaaaa acttgattag ggtgatggtt
  7081 cacgtagtgg gccatcgccc tgatagacgg tttttcgccc tttgacgttg gagtccacgt
  7141 tctttaatag tggactcttg ttccaaactg gaacaacact caaccctatc tcggtctatt
  7201 cttttgattt ataagggatt ttggggattt cggcctattg gttaaaaaat gagctgattt
  7261 aacaaaaatt taacgcgaat taattctgtg gaatgtgtgt cagttagggt gtggaaagtc
  7321 cccaggctcc ccaggcaggc agaagtatgc aaagcatgca tctcaattag tcagcaacca
  7381 ggtgtggaaa gtccccaggc tccccagcag gcagaagtat gcaaagcatg catctcaatt
  7441 agtcagcaac catagtcccg cccctaactc cgcccatccc gcccctaact ccgcccagtt
  7501 ccgcccattc tccgccccat ggctgactaa ttttttttat ttatgcagag gccgaggccg
  7561 cctctgcctc tgagctattc cagaagtagt gaggaggctt ttttggaggc ctaggctttt
  7621 gcaaaaagct cccgggagct tgtatatcca ttttcggatc tgatcagcac gtgttgacaa
  7681 ttaatcatcg gcatagtata tcggcatagt ataatacgac aaggtgagga actaaaccat
  7741 ggccaagttg accagtgccg ttccggtgct caccgcgcgc gacgtcgccg gagcggtcga
  7801 gttctggacc gaccggctcg ggttctcccg ggacttcgtg gaggacgact tcgccggtgt
  7861 ggtccgggac gacgtgaccc tgttcatcag cgcggtccag gaccaggtgg tgccggacaa
  7921 caccctggcc tgggtgtggg tgcgcggcct ggacgagctg tacgccgagt ggtcggaggt
  7981 cgtgtccacg aacttccggg acgcctccgg gccggccatg accgagatcg gcgagcagcc
  8041 gtgggggcgg gagttcgccc tgcgcgaccc ggccggcaac tgcgtgcact tcgtggccga
  8101 ggagcaggac tgacacgtgc tacgagattt cgattccacc gccgccttct atgaaaggtt
  8161 gggcttcgga atcgttttcc gggacgccgg ctggatgatc ctccagcgcg gggatctcat
  8221 gctggagttc ttcgcccacc ccaacttgtt tattgcagct tataatggtt acaaataaag
  8281 caatagcatc acaaatttca caaataaagc atttttttca ctgcattcta gttgtggttt
  8341 gtccaaactc atcaatgtat cttatcatgt ctgtataccg tcgacctcta gctagagctt
  8401 ggcgtaatca tggtcatagc tgtttcctgt gtgaaattgt tatccgctca caattccaca
  8461 caacatacga gccggaagca taaagtgtaa agcctggggt gcctaatgag tgagctaact
  8521 cacattaatt gcgttgcgct cactgcccgc tttccagtcg ggaaacctgt cgtgccagct
  8581 gcattaatga atcggccaac gcgcggggag aggcggtttg cgtattgggc gctcttccgc
  8641 ttcctcgctc actgactcgc tgcgctcggt cgttcggctg cggcgagcgg tatcagctca
  8701 ctcaaaggcg gtaatacggt tatccacaga atcaggggat aacgcaggaa agaacatgtg
  8761 agcaaaaggc cagcaaaagg ccaggaaccg taaaaaggcc gcgttgctgg cgtttttcca
  8821 taggctccgc ccccctgacg agcatcacaa aaatcgacgc tcaagtcaga ggtggcgaaa
  8881 cccgacagga ctataaagat accaggcgtt tccccctgga agctccctcg tgcgctctcc
  8941 tgttccgacc ctgccgctta ccggatacct gtccgccttt ctcccttcgg gaagcgtggc
  9001 gctttctcaa tgctcacgct gtaggtatct cagttcggtg taggtcgttc gctccaagct
  9061 gggctgtgtg cacgaacccc ccgttcagcc cgaccgctgc gccttatccg gtaactatcg
  9121 tcttgagtcc aacccggtaa gacacgactt atcgccactg gcagcagcca ctggtaacag
  9181 gattagcaga gcgaggtatg taggcggtgc tacagagttc ttgaagtggt ggcctaacta
  9241 cggctacact agaaggacag tatttggtat ctgcgctctg ctgaagccag ttaccttcgg
  9301 aaaaagagtt ggtagctctt gatccggcaa acaaaccacc gctggtagcg gtggtttttt
  9361 tgtttgcaag cagcagatta cgcgcagaaa aaaaggatct caagaagatc ctttgatctt
  9421 ttctacgggg tctgacgctc agtggaacga aaactcacgt taagggattt tggtcatgag
  9481 attatcaaaa aggatcttca cctagatcct tttaaattaa aaatgaagtt ttaaatcaat
  9541 ctaaagtata tatgagtaaa cttggtctga cagttaccaa tgcttaatca gtgaggcacc
  9601 tatctcagcg atctgtctat ttcgttcatc catagttgcc tgactccccg tcgtgtagat
  9661 aactacgata cgggagggct taccatctgg ccccagtgct gcaatgatac cgcgagaccc
  9721 acgctcaccg gctccagatt tatcagcaat aaaccagcca gccggaaggg ccgagcgcag
  9781 aagtggtcct gcaactttat ccgcctccat ccagtctatt aattgttgcc gggaagctag
  9841 agtaagtagt tcgccagtta atagtttgcg caacgttgtt gccattgcta caggcatcgt
  9901 ggtgtcacgc tcgtcgtttg gtatggcttc attcagctcc ggttcccaac gatcaaggcg
  9961 agttacatga tcccccatgt tgtgcaaaaa agcggttagc tccttcggtc ctccgatcgt
10021 tgtcagaagt aagttggccg cagtgttatc actcatggtt atggcagcac tgcataattc
10081 tcttactgtc atgccatccg taagatgctt ttctgtgact ggtgagtact caaccaagtc
10141 attctgagaa tagtgtatgc ggcgaccgag ttgctcttgc ccggcgtcaa tacgggataa
10201 taccgcgcca catagcagaa ctttaaaagt gctcatcatt ggaaaacgtt cttcggggcg
10261 aaaactctca aggatcttac cgctgttgag atccagttcg atgtaaccca ctcgtgcacc
10321 caactgatct tcagcatctt ttactttcac cagcgtttct gggtgagcaa aaacaggaag
10381 gcaaaatgcc gcaaaaaagg gaataagggc gacacggaaa tgttgaatac tcatactctt
10441 cctttttcaa tattattgaa gcatttatca gggttattgt ctcatgagcg gatacatatt
10501 tgaatgtatt tagaaaaata aacaaatagg ggttccgcgc acatttcccc gaaaagtgcc
10561 acctgacgtc
```

Although the invention has been described in the context of certain embodiments, it is intended that the patent will not be limited to those embodiment; rather, the scope of this patent shall encompass the full lawful scope of the appended claims, and lawful equivalents thereof.

What is claimed is:

1. A method for facilitating cellular survival of an autologous transplanted or reintroduced cell in an individual having a Type I diabetes or having a pancreatic islet cell disorder, comprising:
(a) providing a stem cell or pancreatic islet cell autologous to the individual, or a stem cell or pancreatic islet cell or a progenitor thereof from the individual;
(b) modifying or genetically engineering the stem cell or pancreatic islet cell or progenitor thereof to enhance or increase the expression of a PIM-1,
wherein modifying or genetically the stem cell or pancreatic islet cell or progenitor thereof to enhance or increase levels of the PIM-1 in the modified or genetically engineered stem cell or pancreatic islet cell or progenitor thereof comprises introducing into the stem cell or pancreatic islet cell or progenitor thereof a PIM-1-encoding nucleic acid;
thereby facilitating cellular survival of the stem cell or pancreatic islet cell or progenitor thereof after transplantation or reintroduction;
and
(c) introducing or reintroducing the modified or genetically engineered PIM-1 enhanced stem cell or pancreatic islet cell or progenitor thereof to the individual or back to the individual having a Type I diabetes or having a pancreatic islet cell disorder.

2. The method of claim 1, wherein the pancreatic islet cell disorder is reduced insulin production or reduced pancreatic islet cell function or numbers.

3. The method of claim 1, wherein the modified or genetically engineered cell is a pancreatic islet cell or a progenitor thereof, or an insulin-producing cell.

4. The method of claim 1, wherein the stem cell autologous to the individual, or the stem cell from the individual is a progenitor cell, a totipotent cell, a pluripotent cell or a multipotent cell; or, a cultured stem cell, progenitor cell, totipotent, pluripotent or multipotent cell.

5. The method of claim 1, wherein the individual is a human or an animal.

6. The method of claim 1, wherein enhancing levels of the PIM-1 in the modified or genetically engineered stem cell or pancreatic islet cell or progenitor thereof comprises enhancing production of an endogenous PIM-1.

7. The method of claim 1, wherein the PIM-1-encoding nucleic acid is a human PIM-1 gene, message or cDNA; or a nucleic acid comprising a sequence as set forth in SEQ ID NO:1, SEQ ID NO:2 or SEQ ID NO:3.

8. The method of claim 1, wherein the PIM-1-encoding nucleic acid is contained in an expression vector, a cosmid, a Yeast Artificial Chromosome, a Mammalian Artificial Chromosome, a viral particle, a retroviral vector, a modified retrovirus having a modified proviral RNA genome, a lentiviral vector, a lentiviral gene therapy vector, a recombinant adeno-associated viral vector, or a phage.

9. The method of claim 8, wherein the PIM-1-encoding nucleic acid, or the expression vector, cosmid, Yeast Artificial Chromosome, Mammalian Artificial Chromosome, viral particle, retroviral vector, modified retrovirus having a modified proviral RNA genome, lentiviral vector, lentiviral gene therapy vector, recombinant adeno-associated viral vector or phage further comprises a suicide sequence,
wherein the suicide sequence can induce apoptosis or otherwise cause cell death upon administration of an exogenous trigger compound or exposure to a trigger, wherein the trigger optionally comprises a light or an electromagnetic radiation exposure.

10. The method of claim 1, wherein the modified or genetically engineered stem cell or pancreatic islet cell or progenitor thereof is enclosed in an immune shielded structure before introduction or re-introduction into the individual.

11. The method of claim 1, wherein the modified or genetically engineered stem cell or pancreatic islet cell or progenitor thereof is introduced or reintroduced into a peritoneal cavity or a kidney capsule of the individual.

12. The method of claim 1, wherein the modified or genetically engineered stem cell is an embryonic stem cell, an endothelial stem cell or a neuronal stem cell.
